(19) United States
(12) Patent Application Publication
(10) Pub. No.: US 2011/0144209 A1
(43) Pub. Date: Jun. 16, 2011

(54) USE OF VASOCONSTRICTORS

(76) Inventor: Oron ZACHAR, Tel-Aviv (IL)

(21) Appl. No.: 12/982,117

(22) Filed: Dec. 30, 2010

Related U.S. Application Data
(63) Continuation-in-part of application No. PCT/IL2009/000652, filed on Jun. 30, 2009.
Application No. PCT/IL2009/000652 is a continuation-in-part of application No. PCT/US2009/066124, filed on Nov. 30, 2009.
(60) Provisional application No. 61/077,056, filed on Jun. 30, 2008.
(60) Provisional application No. 61/079,816, filed on Jul. 11, 2008.
(60) Provisional application No. 61/118,612, filed on Nov. 30, 2008.
(60) Provisional application No. 61/118,612, filed on Nov. 30, 2008.

(30) Foreign Application Priority Data
Jun. 30, 2009 (IL) PCT/IL2009/000652
Nov. 30, 2009 (US) PCT/US2009/066124

(51) Int. Cl. A61K 31/16; A61K 31/135; A61P 35/00

(52) U.S. Cl. 514/616; 514/653

(57) ABSTRACT

There is disclosed the topical dermal use of vasocontrictor substances for regulating body temperature to treat cancer by inducing hyperthermia and treat, prevent or delay the onset of anesthetic induced hypothermia. Kits containing appropriate materials and instructions, and other embodiments, are also disclosed.

This application is a continuation-in-part of PCT/IL2009/000652, filed Jun. 30, 2009, which claims priority from U.S. provisional application No. 61/077,056, filed Jun. 30, 2008; U.S. provisional application No. 61/079,816, filed Jul. 11, 2008; and U.S. provisional application No. 61/118,612, filed Nov. 30, 2008. The benefit of these provisional applications is claimed. This application is also a continuation-in-part of PCT/US2009/066124, filed Nov. 30, 2009, which claims priority from U.S. provisional application No. 61/118,612, filed Nov. 30, 2008, and from PCT/IL2009/000652, filed Jun. 30, 2009. The contents of these applications are incorporated herein by reference.

### BACKGROUND

### A. Present Hyperthermia Treatments for Cancer Patients

Cancer treatment by drug-induced hyperthermia is discussed in the review article “Fever and Cancer in Perspective” by Uwe Hobohm, Cancer Immunol. (2001) 50:391. Hyperthermia can also be achieved generally by the induction of biological fever by pathogens or toxins, or by transfer of heat into the body from an external energy source. A review of known methods is provided in the article “Hyperthermia, systemic” by R. Wanda Rowe-Horwege, published in the Encyclopedia of Medical Devices and Instrumentation, Second Edition (2006), edited by John G. Webster (referred to hereinafter as “Hyperthermia2006”).

As discussed in Hyperthermia2006, typical therapeutic hyperthermia treatments for cancer can be characterized by the following alternative protocols of individual treatment sessions: (i) 1-2 h duration at 41-42° C., or (ii) 3-8 h at 39-40° C. Other temperature range treatments can be interpolated or extrapolated from the above noted treatment examples. Nevertheless, it is believed that temperatures above 38° C. are needed for effective therapeutic hyperthermia treatments for cancer.

Although pyrogenic drugs have been used to induce hyperthermia for the treatment of cancer patients, these drugs have dangerous and undesired effects on patients, as described in the FDA review document titled “PYROGENS, STILL A DANGER”, dated Jan. 12, 1979 and available online at http://www.fda.gov/ICECI/Inspections/InspectionGuides/InspectionTechnicalGuides/ucm072906.htm. The hyperthermia level resulting from administration of pyrogens in a particular individual is difficult to control; and the administration of pyrogens itself generally requires close medical oversight, as pyrogens are generally administered intravenously.

Mechanical heating devices have the advantage that their use is quite safe, and the hyperthermia level and duration are quite tunable per each individual patient. Mechanical heating methods and devices are expensive to use, uncomfortable for the patient, and available for use only on the premises of special treatment institutions. All of which limit their availability and use by wider cancer patient population and limit their coverage by insurance companies.

Hence, there is a need for effective, safe, simple to administer, and relatively cheap methods of inducing hyperthermia for the treatment of cancer patients.

### B. Postoperative Hypothermia (Anesthetic Induced Hypothermia)

The American Society of PeriAnesthesia Nurses defines normothermia as a core body temperature (CBT) ranging between 96.8° F. and 100.4° F. (36° C. and 38° C.), hyperthermia as a CBT greater than 38° C., and hypothermia as a core temperature lower than 96.8° F. (36° C.). Unplanned hypothermia is frequently a problem during or following surgery in which the patient has been administered a general anesthetic. Anesthetic-induced impairment of thermoregulatory control is the primary cause of postoperative hypothermia. Evidence is that the hypothermia starts to develop already during the surgical procedure after the start of administration of anesthesia (Good et al., Association of Operating Room Nurses AORN Journal, May 2006, 83:5, Health Module p. 1055).

Hypothermia has been associated with a number of adverse consequences, including: increased risk of intra-operative blood loss; increased susceptibility to infection (such as myocardial ischemia); impaired coagulation and increased transfusion requirements; cardiovascular stress and cardiac complications; altered drug metabolism; postanesthetic shivering and thermal discomfort, as well as longer patient stays in the postanesthesia care unit (PACU) (Good et al., AORN Journal, May 2006; 83, 5; Health Module p. 1055).

Compared with normothermic patients, patients admitted to the intensive care unit (ICU) with a body core temperature (BCT) <36° C. have a significantly greater mortality, prolonged need for mechanical ventilation, incidence of packed red blood cell transfusion, and prolonged ICU and hospital length of stay (Insler et al., The Annals of thoracic surgery, 2000 July:175).

Methods for preventing anesthetic induced hypothermia commonly employ external heating devices (such as warming blankets, warm air blowers, and the like) and devices to reduce or prevent heat loss, such as light-reflecting covers. Unlike the heating devices used to induce hyperthermia, the heating devices used to treat or prevent anesthetic-induced hypothermia are necessarily designed to allow access to those portions of the patient body undergoing surgery.

Hence, it would be useful if there existed a method to treat or prevent anesthetic-induced hypothermia, which does not involve external devices which may be expensive and/or disturbing to the surgical team.

### BRIEF DESCRIPTION OF EMBODIMENTS OF THE INVENTION

There is provided in accordance with an embodiment of the invention a method for treating a patient having cancer, comprising inducing hyperthermia in the patient by, at least in part, administering to the patient at least one vasoconstrictor. In some embodiments, the administering comprises applying to the skin of a cancer patient the at least one vasoconstrictor. In some embodiments, the hyperthermia is induced in part by at least one of (a) administering to the patient a thermogenic substance (b) applying heat from an external source. In some embodiments, the heat is the ambient heat of the room. In some embodiments, the external source of heat is a warming glove or sock.

In some embodiments, the thermogenic substance is selected from the group consisting of ephedra, bitter orange, capsicum, ginger, caffeine, thyrotropic substances, T3 donor substances, TSH, TRF, VIP, beta-andrenergic agonists, LATS, alpha-2-adrenoreceptor blockers, and ephedrine. In some embodiments, the at least one vasoconstrictor and the thermogenic substance are both active at the same time, and/or the heat is applied at a time when the at least one vasoconstrictor is active.

In some embodiments, the hyperthermia is induced in part by administering to the patient a hypothalamus thermoregulation suppressor. In some embodiments, the hypothalamus thermoregulation suppressor is a selective serotonin uptake inhibitor (SSRI). In some embodiments, the SSRI is MDMA.

In some embodiments, the at least one vasoconstrictor is administered in conjunction with a penentration enhancer. In some embodiments, the at least one vasoconstrictor is dermally applied in conjunction with a penentration enhancer.

In some embodiments, the at least one vasoconstrictor is present in a pharmaceutical composition. In some embodiments, the pharmaceutical composition is a dermally administrable pharmaceutical composition.

In some embodiments, the dermally administrable pharmaceutical composition is applied to at least 10% of the patient's skin. In some embodiments, the dermally administrable pharmaceutical composition is applied to at least 15% of the patient's skin. In some embodiments, the dermally administrable pharmaceutical composition is applied to at least 20% of the patient's skin. In some embodiments, the dermally administrable pharmaceutical composition is applied to at least 25% of the patient's skin. In some embodiments, the dermally administrable pharmaceutical composition is applied to at least 30% of the patent's skin. In some embodiments, the dermally administrable pharmaceutical composition is applied to at least 35% of the patent's skin.

In some embodiments, the skin to which the at least one vasoconstrictor is applied is selected from the group consisting of the palm of the hand, the sole of the foot, the ear, and the face.

In some embodiments, the vasoconstrictor is applied in the form of a dermally administrable pharmaceutical composition containing at least 1.0% by weight of the at least one vasoconstrictor or mixture of vasoconstrictors. In some embodiments, the dermally administrable pharmaceutical composition contains at least 2.0% by weight of the at least one vasoconstrictor. In some embodiments, the dermally administrable pharmaceutical composition contains at least 3.0% by weight of the at least one vasoconstrictor. In some embodiments, the dermally administrable pharmaceutical composition contains at least 4.0% by weight of the at least one vasoconstrictor. In some embodiments, the dermally administrable pharmaceutical composition contains at least 5.0% by weight of the at least one vasoconstrictor. In some embodiments, the dermally administrable pharmaceutical composition contains at least 5.0% by weight of the at least one vasoconstrictor. In some embodiments, the dermally administrable pharmaceutical composition contains at least 10.0% by weight of the at least one vasoconstrictor. In some embodiments, the dermally administrable pharmaceutical composition contains at least 15.0% by weight of the at least one vasoconstrictor. In some embodiments, the dermally administrable pharmaceutical composition contains at least 20.0% by weight of the at least one vasoconstrictor. In some embodiments, the dermally administrable pharmaceutical composition contains at least 25.0% by weight of the at least one vasoconstrictor. In some embodiments, the dermally administrable pharmaceutical composition contains at least 30.0% by weight of the at least one vasoconstrictor.

In some embodiments, at least two vasoconstrictors are used, each of the at least two vasoconstrictors having peak effectiveness at different times after administration.

In some embodiments, the at least one vasoconstrictor is selected from the group consisting of (i) the group consisting of vasoactive agonists, vasopressor agents and vasoconstrictor drugs; (ii) an agent that acts on vasopressin receptors or adrenoreceptors; (iii) a calcium channel agonist; (iv) an agonist of the α₁ adrenergic receptor; (v) alfuzosin, doxazosin, epinephrine, methoxamine, naphazoline, norepinephrine, phenylephrine, prazosin, terazosin, tetrahydrozaline, tamsulosin; (vi) an agonist of the 5HT₁B_{A}D receptor; (vii) almotriptan, avitriptan, frovatriptan, oxidesumitriptan, rizatriptan, zolmitriptan; (viii) chlorpheniramine, ethylnorepinephrine, mephenterine, metaraminol, oxymetazoline, oxymetazoline, phenylpropanolamine, potassium chloride, pseudoephidrine, propylhexadrine; (ix) ephedrine, angiotensin and vasopressin; (x) tetrahydrozoline HCl 0.05%, naphazoline HCl 0.03%, oxymetazoline HCl 0.025%; (xi) a vasoconstrictor extract selected from the group including ephedra sinica (ma huang), polygonum bistorta (bistort root), hamamelis virginiana (witch hazel), hydrastis canadensis (goldenseal), lycopus virginicus (bugleweed), aspidosperma quebracho (quebracho bianco), cytisus scoparius (scotch broom), guava extract, ellagic acid, caffeine, peppermint extract, chamomile oil, and cypress; (xii) an agent that positively affects the McKenzie vasoconstrictor assay; (xiii) topical corticosteroids, hydrocortisone, cortisol, synthetic corticosteroids, betametasone, fluticasone, mometasone.

In some embodiments, the at least one vasoconstrictor is selected from the group consisting of methoxamine, methylnorepinephrine, oxymetazoline, phenylephrine, metaraminol, 4-(1-naphthalen-1-ylethyl)-1H-imidazole (4-NEMD), clonidine, guanfacine, guanabenz, guanoxabenz, guanethidine, xylazine, methyldopa, apraclonidine, brimonidine, detomidine, dexmedetomidine, lofexidine, romifidine, tizanidine, xylometazoline, amidephrine, amitraz, anisodamine, ergotamine, indanidine, medetomidine, mephentermine, midodrine, mivazerol, naphazoline, norfenefrine, octopamine, phenylpropanolamine, rilmenidine, synephrine, talipexole, tetrahydrozoline, xylometazoline, dobutamine, dopamine, denopamine, xamoterol, salbutamol, levosalbutamol, fenoterol, terbutaline, pirbuterol, procaterol, bitolterol, rimiterol, carbuterol, tulobuterol, reproterol, dopexamine, arformoterol, bambuterol, clenbuterol, formoterol, salmeterol, orciprenaline, metaproterenol, ritodrine, hexoprenaline, indacaterol, amibegron, solabegron, arbutamine, befunolol, isoxsuprine, nylidrin, oxyfedrine, prenalterol, ractopamine, bromoacetylalprenololmenthane, broxaterol, cimaterol, higenamine, mabuterol, methoxyphenamine, tretoquinol, zinterol isoprenaline, isoproterenol, epinephrine, norepinephrine, cirazoline, etilefrine, amphetamine, tyramine, ephedrine, pseudoephedrine, cocaine, allobarbital, amobarbital, aprobarbital, barbital, butobarbital, cyclobarbital, ethallobarbital, heptabarbital, hexobarbital, methohexital, pentobarbital, phenobarbital, proxibarbal, reposal, secobarbital, talbutal, thiopental, vinylbital, vinbarbital, brotizolam, cinolazepam, doxefazepam, estazolam, flunitrazepam, flurazepam, flutoprazepam, loprazolam, lormetazepam, nitrazepam, nimetazepam, midazolam, quazepam, temazepam, triazolam CL-218872, eszopiclone, indiplon, necopidem, pazinaclone, ROD-188, saripidem, suproclone, suriclone, SX-3228, U-89843A, U-90042, zaleplon, zolpidem, zopiclone, glutethimide, methyprylon, pyrithyldione afloqualone, cloroqualone, diproqualone, etaqualone, mebroqualone, mecloqualone, methaqualone, methylmethaqualone acebrochol, allopregnanolone, alphadolone, alphaxolone, ganaxolone, hydroxydione, minaxolone, Org 20599, tetrahydrodeoxycorticosterone, dexmedetomidine, lofexidine, medetomidine, romifidine, tizanidine, xylazine agomelatine, melatonin, ramelteon, doxylamine, hydroxyzine, diphenhydramine, bromodiphenhydramine, carbinoxamine, orphenadrine, niaprazine, phenyltoloxamine, propiomazine, pyrilamine, scopolamine, aceburic acid, gamma-amino-beta-hydroxybutyric acid (GABOB), gamma-hydroxybutyric acid (GHB), sodium oxybate, Xyrem®, gamma-butyrolactone (GBL), 1,4-butanediol, 3-chloropropanoic acid, acetylglycinamide chloral hydrate, chloral hydrate, chloralodol, dichloralphenazone, paraldehyde, petrichloral, centalun, ethchlorvynol, ethinamate, hexapropymate, methylpentynol, meprobamate, carisoprodol, tybamate, methocarbamol, 2-methyl-2-butanol, acecarbromal, apronal, bromisoval, carbromal, clomethiazole, embutramide, etomidate, gaboxadol, loreclezole, mephenoxalone, sulfonmethane, trichloroethanol, triclofos, valerian, valnoctamide and trazadone.

In some embodiments, the at least one vasoconstrictor is applied in the form of a dermally administrable pharmaceutical composition that further comprises a substance selected from the group consisting of a penetration enhancer, an antiseptic compound, an antibiotic compound, an antimycotic compound, and an antiviral compound.

In some embodiments, the at least one vasoconstrictor is applied at a concentration and/or to an amount of skin effective, in conjunction with the administration of the thermogenic compound and/or the application of heat from an external source, to raise the patient's core body temperature to a temperature of at least 38.5° C. for a period of at least 60 minutes. In some embodiments, the at least one vasoconstrictor is applied at a concentration and/or to an amount of skin effective, in conjunction with the administration of the thermogenic compound and/or the application of heat from an external source, to raise the patient's core body temperature to a temperature of 41-42° C. for a period of one to two hours. In some embodiments, the at least one vasoconstrictor is applied at a concentration and/or to an amount of skin effective, in conjunction with the administration of the thermogenic compound and/or the application of heat from an external source, to raise the patient's core body temperature to a temperature of 39-40° C. for a period of at three to eight hours.

In some embodiments, the patient is not undergoing chemotherapy. In some embodiments, the patient is not undergoing radiation therapy. In some embodiments, patient is not suffering from side effects of chemo- or radiation therapy.

In some embodiments, the method further comprises administering to the patient diosmin.

In some embodiments, the method further comprises administering a vasodilator to reduce the patient's temperature.

In some embodiments, the ambient humidity is at least 50%. In some embodiments, the ambient humidity is at least 60%. In some embodiments, the ambient humidity is at least 70%. In some embodiments, the ambient humidity is at least 75%. In some embodiments, the ambient humidity is at least 80%.

In some embodiments, the method further comprises administering an antiperspirant to the patient.

There is also provided, in accordance with an embodiment of the invention, a kit comprising at least one vasoconstrictor and instructions or a label explaining how to use the at least one vasoconstrictor to treat cancer by inducing hyperthermia in the patient by, at least in part, administering the vasoconstrictor a cancer patient. In some embodiments, the administering comprises applying the at least one vasoconstrictor to the skin of a cancer patient. In some embodiments, the vasoconstrictor is present in a dermally administrable pharmaceutical composition, and the instructions or label instruct the user to apply the dermally administrable pharmaceutical composition to at least 10% of the patient's skin. In some embodiments, the instructions or label instruct the user to apply the dermally administrable pharmaceutical composition to at least 15% of the patient's skin. In some embodiments, the instructions or label instruct the user to apply the dermally administrable pharmaceutical composition to at least 20% of the patient's skin. In some embodiments, the instructions or label instruct the user to apply the dermally administrable pharmaceutical composition to at least 25% of the patient's skin. In some embodiments, the instructions or label instruct the user to apply the dermally administrable pharmaceutical composition to at least 30% of the patient's skin. In some embodiments, the instructions or label instruct the user to apply the dermally administrable pharmaceutical composition to at least 35% of the patient's skin.

In some embodiments, the instructions or label instruct the user how to prepare a dermally administrable pharmaceutical composition containing the at least one vasoconstrictor and to apply the pharmaceutical composition to the patient's skin.

In some embodiments, the instructions or label instruct that the skin to which the at least one vasoconstrictor is applied includes skin selected from the group consisting of the palm of the hand, the sole of the foot, the ear, and the face.

In some embodiments, the vasoconstrictor is supplied in the form of a dermally administrable pharmaceutical composition containing at least 1.0% by weight of the at least one vasoconstrictor. In some embodiments, the dermally administrable pharmaceutical composition contains at least 2.0% by weight of the at least one vasoconstrictor. In some embodiments, the dermally administrable pharmaceutical composition contains at least 3.0% by weight of the at least one vasoconstrictor. In some embodiments, the dermally administrable pharmaceutical composition contains at least 3.0% by weight of the at least one vasoconstrictor. In some embodiments, the dermally administrable pharmaceutical composition contains at least 4.0% by weight of the at least one vasoconstrictor. In some embodiments, the dermally administrable pharmaceutical composition contains at least 5.0% by weight of the at least one vasoconstrictor. In some embodiments, the dermally administrable pharmaceutical composition contains at least 10.0% by weight of the at least one vasoconstrictor. In some embodiments, the dermally administrable pharmaceutical composition contains at least 15.0% by weight of the at least one vasoconstrictor. In some embodiments, the dermally administrable pharmaceutical composition contains at least 20.0% by weight of the at least one vasoconstrictor. In some embodiments, the dermally administrable pharmaceutical composition contains at least 25.0% by weight of the at least one vasoconstrictor. In some embodiments, the dermally administrable pharmaceutical composition contains at least 30.0% by weight of the at least one vasoconstrictor.

In some embodiments, at least two vasoconstrictors are used, each of the at least two vasoconstrictors having peak effectiveness at different times after administration.

In some embodiments, the at least one vasoconstrictor is selected from the group consisting of (i) the group consisting of vasoactive agonists, vasopressor agents and vasoconstrictor drugs; (ii) an agent that acts on vasopressin receptors or adrenoreceptors; (iii) a calcium channel agonist; (iv) an agonist of the α₁ adrenergic receptor; (v) alfuzosin, doxazosin, epinephrine, methoxamine, naphazoline, norepinephrine, phenylephrine, prazosin, terazosin, tetrahydrozaline, tamsulosin; (vi) an agonist of the 5HT₁B_{A}D receptor; (vii) almotriptan, avitriptan, frovatriptan, oxidesumitriptan, rizatriptan, zolmitriptan; (viii) chlorpheniramine, ethylnorepinephrine, mephenterine, metaraminol, oxymetazoline, oxymetazoline, phenylpropanolamine, potassium chloride, pseudoephidrine, propylhexadrine; (ix) ephedrine, angiotensin and vasopressin; (x) tetrahydrozoline HCl 0.05%, naphazoline HCl 0.03%, oxymetazoline HCl 0.025%; (xi) a vasoconstrictor extract selected from the group including ephedra sinica (ma huang), polygonum bistorta (bistort root), hamamelis virginiana (witch hazel), hydrastis canadensis (goldenseal), lycopus virginicus (bugleweed), aspidosperma quebracho (quebracho bianco), cytisus scoparius (scotch broom), guava extract, ellagic acid, caffeine, peppermint extract, chamomile oil, and cypress; (xii) an agent that positively affects the McKenzie vasoconstrictor assay; (xiii) topical corticosteroids, hydrocortisone, cortisol, synthetic corticosteroids, betametasone, fluticasone, mometasone.

In some embodiments, the at least one vasoconstrictor is selected from the group consisting of methoxamine, methylnorepinephrine, oxymetazoline, phenylephrine, metaraminol, 4-(1-naphthalen-1-ylethyl)-1H-imidazole (4-NEMD), clonidine, guanfacine, guanabenz, guanoxabenz, guanethidine, xylazine, methyldopa, apraclonidine, brimonidine, detomidine, dexmedetomidine, lofexidine, romifidine, tizanidine, xylometazoline, amidephrine, amitraz, anisodamine, ergotamine, indanidine, medetomidine, mephentermine, midodrine, mivazerol, naphazoline, norfenefrine, octopamine, phenylpropanolamine, rilmenidine, synephrine, talipexole, tetrahydrozoline, xylometazoline, dobutamine, dopamine, denopamine, xamoterol, salbutamol, levosalbutamol, fenoterol, terbutaline, pirbuterol, procaterol, bitolterol, rimiterol, carbuterol, tulobuterol, reproterol, dopexamine, arformoterol, bambuterol, clenbuterol, formoterol, salmeterol, orciprenaline, metaproterenol, ritodrine, hexoprenaline, indacaterol, amibegron, solabegron, arbutamine, befunolol, isoxsuprine, nylidrin, oxyfedrine, prenalterol, ractopamine, bromoacetylalprenololmenthane, broxaterol, cimaterol, higenamine, mabuterol, methoxyphenamine, tretoquinol, zinterol isoprenaline, isoproterenol, epinephrine, norepinephrine, cirazoline, etilefrine, amphetamine, tyramine, ephedrine, pseudoephedrine, cocaine, allobarbital, amobarbital, aprobarbital, barbital, butobarbital, cyclobarbital, ethallobarbital, heptabarbital, hexobarbital, methohexital, pentobarbital, phenobarbital, proxibarbal, reposal, secobarbital, talbutal, thiopental, vinylbital, vinbarbital, brotizolam, cinolazepam, doxefazepam, estazolam, flunitrazepam, flurazepam, flutoprazepam, loprazolam, lormetazepam, nitrazepam, nimetazepam, midazolam, quazepam, temazepam, triazolam CL-218872, eszopiclone, indiplon, necopidem, pazinaclone, ROD-188, saripidem, suproclone, suriclone, SX-3228, U-89843A, U-90042, zaleplon, zolpidem, zopiclone, glutethimide, methyprylon, pyrithyldione afloqualone, cloroqualone, diproqualone, etaqualone, mebroqualone, mecloqualone, methaqualone, methylmethaqualone acebrochol, allopregnanolone, alphadolone, alphaxolone, ganaxolone, hydroxydione, minaxolone, Org 20599, tetrahydrodeoxycorticosterone, dexmedetomidine, lofexidine, medetomidine, romifidine, tizanidine, xylazine agomelatine, melatonin, ramelteon, doxylamine, hydroxyzine, diphenhydramine, bromodiphenhydramine, carbinoxamine, orphenadrine, niaprazine, phenyltoloxamine, propiomazine, pyrilamine, scopolamine, aceburic acid, gamma-amino-beta-hydroxybutyric acid (GABOB), gamma-hydroxybutyric acid (GHB), sodium oxybate, Xyrem®, gamma-butyrolactone (GBL), 1,4-butanediol, 3-chloropropanoic acid, acetylglycinamide chloral hydrate, chloral hydrate, chloralodol, dichloralphenazone, paraldehyde, petrichloral, centalun, ethchlorvynol, ethinamate, hexapropymate, methylpentynol, meprobamate, carisoprodol, tybamate, methocarbamol, 2-methyl-2-butanol, acecarbromal, apronal, bromisoval, carbromal, clomethiazole, embutramide, etomidate, gaboxadol, loreclezole, mephenoxalone, sulfonmethane, trichloroethanol, triclofos, valerian, valnoctamide and trazadone.

In some embodiments, the at least one vasoconstrictor is provided in the form of a dermally administrable pharmaceutical composition that further comprises a substance selected from the group consisting of a penetration enhancer, an antiseptic compound, an antibiotic compound, an antimycotic compound, and an antiviral compound.

In some embodiments, the instructions or label explain how to administer the at least one vasoconstrictor effective, in conjunction with the administration of the thermogenic compound and/or the application of heat from an external source, to raise the patient's core body temperature by at least 1° C. within a period of 1 hour. In some embodiments, the instructions or label explain how to administer the at least one vasoconstrictor effective, in conjunction with the administration of the thermogenic compound and/or the application of heat from an external source, to raise the patient's core body temperature to a temperature of at least 38.5° C. for a period of at least 60 minutes. In some embodiments, the instructions or label explain how to administer the at least one vasoconstrictor effective, in conjunction with the administration of the thermogenic compound and/or the application of heat from an external source, to raise the patient's core body temperature to a temperature of 41-42° C. for a period of one to two hours. In some embodiments, the instructions or label explain how to administer the at least one vasoconstrictor effective, in conjunction with the administration of the thermogenic compound and/or the application of heat from an external source, to raise the patient's core body temperature to a temperature of 39-40° C. for a period of at three to eight hours. In some embodiments, the instructions or label explain how to apply the at least one vasoconstrictor at a concentration and/or to an amount of skin effective, in conjunction with the administration of the thermogenic compound and/or the application of heat from an external source, to raise the patient's core body temperature by at least 1° C. within a period of 1 hour. In some embodiments, the instructions or label explain how to apply the at least one vasoconstrictor at a concentration and/or to an amount of skin effective, in conjunction with the administration of the thermogenic compound and/or the application of heat from an external source, to raise the patient's core body temperature to a temperature of at least 38.5° C. for a period of at least 60 minutes. In some embodiments, the instructions or label explain how to apply the at least one vasoconstrictor at a concentration and/or to an amount of skin effective, in conjunction with the administration of the thermogenic compound and/or the application of heat from an external source, to raise the patient's core body temperature to a temperature of 41-42° C. for a period of one to two hours. In some embodiments, the instructions or label explain how to apply the at least one vasoconstrictor at a concentration and/or to an amount of skin effective, in conjunction with the administration of the thermogenic compound and/or the application of heat from an external source, to raise the patient's core body temperature to a temperature of 39-40° C. for a period of at three to eight hours.

In some embodiments, the patient is at least one of (a) not undergoing chemotherapy, (b) not undergoing radiation therapy, and (c) not suffering from side effects of chemo- or radiation therapy.

In some embodiments, the instructions or label further explain how to use diosmin in conjunction with the at least one vasoconstrictor. In some embodiments, the kit further comprises diosmin.

In some embodiments, the kit further comprises a vasodilator. In some embodiments, the instructions or label further explain how to use a vasodilator to reduce the patient's temperature after the administration of the at least one vasoconstrictor.

In some embodiments, the instructions or label further instruct to apply the at least one vasoconstrictor under ambient humidity of at least 50%. In some embodiments, the instructions or label further instruct to apply the at least one vasoconstrictor under ambient humidity of at least 60%. In some embodiments, the instructions or label further instruct to apply the at least one vasoconstrictor under ambient humidity of at least 70%. In some embodiments, the instructions or label further instruct to apply the at least one vasoconstrictor under ambient humidity of at least 75%. In some embodiments, the instructions or label further instruct to apply the at least one vasoconstrictor under ambient humidity of at least 80%.

In some embodiments, the instructions or label further instruct to administer an antiperspirant to the patient.

There also is provided, in accordance with an embodiment of the invention, a method for treating, preventing or delaying the onset of anesthetic hypothermia, which comprises administering to a patient who is under general anesthetic or is about to be put under general anesthetic an amount of at least one vasoconstrictor effective to treat, prevent or delay the onset of anesthetic hypothermia. In some embodiments, the administering comprising applying the at least one vasoconstrictor to the skin of the patient. In some embodiments, the at least one vasoconstrictor is present as a mixture of vasoconstrictors. In some embodiments, the vasoconstrictor or mixture of vasoconstrictors is present in a pharmaceutical composition. In some embodiments, the pharmaceutical composition is a dermally administrable pharmaceutical composition. In some embodiments, the dermally administrable pharmaceutical composition is applied to at least 10% of the patient's skin. In some embodiments, the dermally administrable pharmaceutical composition is applied to at least 15% of the patient's skin. In some embodiments, the dermally administrable pharmaceutical composition is applied to at least 20% of the patient's skin. In some embodiments, the dermally administrable pharmaceutical composition is applied to at least 25% of the patient's skin. In some embodiments, the dermally administrable pharmaceutical composition is applied to at least 30% of the patient's skin. In some embodiments, the dermally administrable pharmaceutical composition is applied to at least 35% of the patient's skin. In some embodiments, the dermally administrable pharmaceutical composition is applied to at least 40% of the patient's skin. In some embodiments, the dermally administrable pharmaceutical composition is applied to at least 45% of the patient's skin. In some embodiments, the dermally administrable pharmaceutical composition is applied to at least 50% of the patient's skin.

In some embodiments, the skin to which the at least one vasoconstrictor is applied includes skin selected from the group consisting of the palm of the hand, the sole of the foot, the ear, and the face. In some embodiments, the at least one vasoconstrictor is applied in the form of a dermally administrable pharmaceutical composition containing at least 1% by weight of the at least one vasoconstrictor. In some embodiments, the composition contains at least 2% by weight of the at least one vasoconstrictor. In some embodiments, the composition contains at least 3% by weight of the at least one vasoconstrictor. In some embodiments, the composition contains at least 4% by weight of the at least one vasoconstrictor. In some embodiments, the composition contains at least 5% by weight of the at least one vasoconstrictor. In some embodiments, the composition contains at least 10% by weight of the at least one vasoconstrictor. In some embodiments, the composition contains at least 20% by weight of the at least one vasoconstrictor. In some embodiments, the composition contains at least 25% by weight of the at least one vasoconstrictor. In some embodiments, the composition contains at least 30% by weight of the at least one vasoconstrictor.

In some embodiments, the at least one vasoconstrictor is administered in the form of a pharmaceutical composition containing at least 1% by weight of the at least one vasoconstrictor. In some embodiments, the composition contains at least 2% by weight of the at least one vasoconstrictor. In some embodiments, the composition contains at least 3% by weight of the at least one vasoconstrictor. In some embodiments, the composition contains at least 4% by weight of the at least one vasoconstrictor. In some embodiments, the composition contains at least 5% by weight of the at least one vasoconstrictor. In some embodiments, the composition contains at least 10% by weight of the at least one vasoconstrictor. In some embodiments, the composition contains at least 20% by weight of the at least one vasoconstrictor. In some embodiments, the composition contains at least 25% by weight of the at least one vasoconstrictor. In some embodiments, the composition contains at least 30% by weight of the at least one vasoconstrictor

In some embodiments, at least two vasoconstrictors are used, each of the at least two vasoconstrictors having peak effectiveness at different times after administration. In some embodiments, at least two vasoconstrictors are used, each of the at least two vasoconstrictors exerting its vasoconstrictive effect via a different mechanism. In some embodiments, at least two vasoconstrictors are used, each of the at least two vasoconstrictors having a different duration of effectiveness after administration.

In some embodiments, at least one vasoconstrictor is selected from the group consisting of (i) the group consisting of vasoactive agonists, vasopressor agents and vasoconstrictor drugs; (ii) an agent that acts on vasopressin receptors or adrenoreceptors; (iii) a calcium channel agonist; (iv) an agonist of the α₁ adrenergic receptor; (v) alfuzosin, doxazosin, epinephrine, methoxamine, naphazoline, norepinephrine, phenylephrine, prazosin, terazosin, tetrahydrozaline, tamsulosin; (vi) an agonist of the 5HT₁B_{A}D receptor; (vii) almotriptan, avitriptan, frovatriptan, oxidesumitriptan, rizatriptan, zolmitriptan; (viii) chlorpheniramine, ethylnorepinephrine, mephenterine, metaraminol, oxymetazoline, oxymetazoline, phenylpropanolamine, potassium chloride, pseudoephidrine, propylhexadrine; (ix) ephedrine, angiotensin and vasopressin; (x) tetrahydrozoline HCl 0.05%, naphazoline HCl 0.03%, oxymetazoline HCl 0.025%; (xi) a vasoconstrictor extract selected from the group including ephedra sinica (ma huang), polygonum bistorta (bistort root), hamamelis virginiana (witch hazel), hydrastis canadensis (goldenseal), lycopus virginicus (bugleweed), aspidosperma quebracho (quebracho bianco), cytisus scoparius (scotch broom), guava extract, ellagic acid, caffeine, peppermint extract, chamomile oil, and cypress; (xii) an agent that positively affects the McKenzie vasoconstrictor assay; (xiii) topical corticosteroids, hydrocortisone, cortisol, synthetic corticosteroids, betametasone, fluticasone, mometasone; (xiv) antagonists of the β₂ adrenergic receptor, such as butaxamine.

In some embodiments, the at least one vasoconstrictor is selected from the group consisting of methoxamine, methylnorepinephrine, oxymetazoline, phenylephrine, metaraminol, 4-(1-naphthalen-1-ylethyl)-1H-imidazole (4-NEMD), clonidine, guanfacine, guanabenz, guanoxabenz, guanethidine, xylazine, methyldopa, apraclonidine, brimonidine, detomidine, dexmedetomidine, lofexidine, romifidine, tizanidine, xylometazoline, amidephrine, amitraz, anisodamine, ergotamine, indanidine, medetomidine, mephentermine, midodrine, mivazerol, naphazoline, norfenefrine, octopamine, phenylpropanolamine, rilmenidine, synephrine, talipexole, tetrahydrozoline, xylometazoline, dobutamine, dopamine, denopamine, xamoterol, salbutamol, levosalbutamol, fenoterol, terbutaline, pirbuterol, procaterol, bitolterol, rimiterol, carbuterol, tulobuterol, reproterol, dopexamine, arformoterol, bambuterol, clenbuterol, formoterol, salmeterol, orciprenaline, metaproterenol, ritodrine, hexoprenaline, indacaterol, amibegron, solabegron, arbutamine, befunolol, isoxsuprine, nylidrin, oxyfedrine, prenalterol, ractopamine, bromoacetylalprenololmenthane, broxaterol, cimaterol, higenamine, mabuterol, methoxyphenainine, tretoquinol, zinterol isoprenaline, isoproterenol, epinephrine, norepinephrine, cirazoline, etilefrine, amphetamine, tyramine, ephedrine, pseudoephedrine, cocaine, allobarbital, amobarbital, aprobarbital, barbital, butobarbital, cyclobarbital, ethallobarbital, heptabarbital, hexobarbital, methohexital, pentobarbital, phenobarbital, proxibarbal, reposal, secobarbital, talbutal, thiopental, vinylbital, vinbarbital, brotizolam, cinolazepam, doxefazepam, estazolam, flunitrazepam, flurazepam, flutoprazepam, loprazolam, lormetazepam, nitrazepam, nimetazepam, midazolam, quazepam, temazepam, triazolam CL-218872, eszopiclone, indiplon, necopidem, pazinaclone, ROD-188, saripidem, suproclone, suriclone, SX-3228, U-89843A, U-90042, zaleplon, zolpidem, zopiclone, glutethimide, methyprylon, pyrithyldione afloqualone, cloroqualone, diproqualone, etaqualone, mebroqualone, mecloqualone, methaqualone, methylmethaqualone acebrochol, allopregnanolone, alphadolone, alphaxolone, ganaxolone, hydroxydione, minaxolone, Org 20599, tetrahydrodeoxycorticosterone, dexmedetomidine, lofexidine, medetomidine, romifidine, tizanidine, xylazine agomelatine, melatonin, ramelteon, doxylamine, hydroxyzine, diphenhydramine, bromodiphenhydramine, carbinoxamine, orphenadrine, niaprazine, phenyltoloxamine, propiomazine, pyrilamine, scopolamine, aceburic acid, gamma-amino-beta-hydroxybutyric acid (GABOB), gamma-hydroxybutyric acid (GHB), sodium oxybate, Xyrem®, gamma-butyrolactone (GBL), 1,4-butanediol, 3-chloropropanoic acid, acetylglycinamide chloral hydrate, chloral hydrate, chloralodol, dichloralphenazone, paraldehyde, petrichloral, centalun, ethchlorvynol, ethinamate, hexapropymate, methylpentynol, meprobamate, carisoprodol, tybamate, methocarbamol, 2-methyl-2-butanol, acecarbromal, apronal, bromisoval, carbromal, clomethiazole, embutramide, etomidate, gaboxadol, loreclezole, mephenoxalone, sulfonmethane, trichloroethanol, triclofos, valerian, valnoctamide and trazadone. In some embodiments, the at least one vasoconstrictor is selected from the group consisting of α₁ agonists and β₂ blockers. In some embodiments, the at least one vasoconstrictor is selected from the group consisting of epinephrine, norepinephrine, and pharmaceutically acceptable salts thereof.

In some embodiments, the at least one vasoconstrictor is applied in the form of a dermally administrable pharmaceutical composition that further comprises at least one of an antiseptic, antibiotic, antimycotic, or antiviral compound. In some embodiments, the at least one vasoconstrictor is applied in the form of a dermally administrable pharmaceutical composition that further comprises a penetration enhancer.

In some embodiments, the at least one vasoconstrictor is administered in an amount effective to raise the patient's core body temperature by 1° C. within a period of 60 minutes from administration. In some embodiments, the at least one vasoconstrictor is administered in an amount effective to raise the patient's core body temperature by at least 1° C. for a period of at least 1 hour. In some embodiments, the at least one vasoconstrictor is administered in conjunction with at least one of (a) a penetration enhancer, (b) heat from an external source, and (c) a thermogenic substance. In some embodiments, one vasoconstrictor is used. In some embodiments, more than one vasoconstrictor is used. In some embodiments, a mixture of vasoconstrictors is used. In some embodiments, the at least one vasoconstrictor is administered by application to the patient's skin, wherein the at least one vasoconstrictor is applied at a concentration and/or to an amount of skin effective to raise the patient's core body temperature by 1° C. within a period of 60 minutes from administration. In some embodiments, the at least one vasoconstrictor is administered by application to the patient's skin, wherein the at least one vasoconstrictor is applied at a concentration and/or to an amount of skin effective to raise the patient's core body temperature by at least 1° C. for a period of at least 1 hour. In some embodiments, the at least one vasoconstrictor is administered by application to the patient's skin, wherein the at least one vasoconstrictor is applied in conjunction with at least one of (a) a penetration enhancer, (b) heat from an external source, and (c) a thermogenic substance. In some embodiments, one vasoconstrictor is used. In some embodiments, more than one vasoconstrictor is used. In some embodiments, a mixture of vasoconstrictors is used.

There is also provided, in accordance with an embodiment of the invention, a kit comprising at least one vasoconstrictor and instructions or a label explaining how to use the at least one vasoconstrictor to treat, prevent or delay the onset of anesthetic hypothermia in a patient. In some embodiments, the instructions or label instruct the user to administer the at least one vasoconstrictor to the skin of a patient who is under general anesthetic or is about to be put under general anesthetic in an amount effective to treat, prevent or delay the onset of anesthetic hypothermia. In some embodiments, the instructions or label instruct the user to apply the at least one vasoconstrictor to the skin of a patient who is under general anesthetic or is about to be put under general anesthetic in an amount effective to treat, prevent or delay the onset of anesthetic hypothermia. In some embodiments, the vasoconstrictor is present in a pharmaceutical composition. In some embodiments, pharmaceutical composition is a dermally administrable pharmaceutical composition. In some embodiments, the instructions or label instruct the user how to prepare a pharmaceutical composition containing the at least one vasoconstrictor. In some embodiments, the instructions or label instruct the user how to prepare a dermally administrable pharmaceutical composition containing the at least one vasoconstrictor. In some embodiments, the dermally administrable pharmaceutical composition contains at least 1% by weight of the at least one vasoconstrictor. In some embodiments, the dermally administrable pharmaceutical composition contains at least 2% by weight of the at least one vasoconstrictor. In some embodiments, the dermally administrable pharmaceutical composition contains at least 3% by weight of the at least one vasoconstrictor. In some embodiments, the dermally administrable pharmaceutical composition contains at least 4% by weight of the at least one vasoconstrictor. In some embodiments, the dermally administrable pharmaceutical composition contains at least 5% by weight of the at least one vasoconstrictor. In some embodiments, the dermally administrable pharmaceutical composition contains at least 10% by weight of the at least one vasoconstrictor. In some embodiments, the dermally administrable pharmaceutical composition contains at least 15% by weight of the at least one vasoconstrictor. In some embodiments, the dermally administrable pharmaceutical composition contains at least 20% by weight of the at least one vasoconstrictor. In some embodiments, the dermally administrable pharmaceutical composition contains at least 25% by weight of the at least one vasoconstrictor. In some embodiments, the dermally administrable pharmaceutical composition contains at least 30% by weight of the at least one vasoconstrictor. In some embodiments, the instructions or label instruct the user and to apply the dermally administrable pharmaceutical composition to at least 10% of the patient's skin. In some embodiments, the instructions or label instruct the user and to apply the dermally administrable pharmaceutical composition to at least 15% of the patient's skin. In some embodiments, the instructions or label instruct the user and to apply the dermally administrable pharmaceutical composition to at least 20% of the patient's skin. In some embodiments, the instructions or label instruct the user and to apply the dermally administrable pharmaceutical composition to at least 25% of the patient's skin. In some embodiments, the instructions or label instruct the user to apply the dermally administrable pharmaceutical composition to at least 30% of the patient's skin. In some embodiments, the instructions or label instruct the user and to apply the dermally administrable pharmaceutical composition to at least 35% of the patient's skin. In some embodiments, the instructions or label instruct the user and to apply the dermally administrable pharmaceutical composition to at least 40% of the patient's skin. In some embodiments, the instructions or label instruct the user and to apply the dermally administrable pharmaceutical composition to at least 45% of the patient's skin. In some embodiments, the instructions or label instruct the user and to apply the dermally administrable pharmaceutical composition to at least 50% of the patient's skin. In some embodiments, the instructions or label instruct that the skin to which the vasoconstrictor is applied includes skin selected from the group consisting of the palm of the hand, the sole of the foot, the ear, and the face. In some embodiments, the composition further comprises a substance selected from the group consisting of a penetration enhancer, an antiseptic compound, an antibiotic compound, an antimycotic compound, and an antiviral compound. In some embodiments, the kit comprises at least two vasoconstrictors, each of the at least two vasoconstrictors having peak effectiveness at different times after administration. In some embodiments, the kit comprises at least two vasoconstrictors, wherein each of the at least two vasoconstrictors exerts its vasoconstrictive effect via a different mechanism. In some embodiments, the kit comprises at least two vasoconstrictors, each of the at least two vasoconstrictors having a different duration of effectiveness after administration.

In some embodiments, the at least one vasoconstrictor is selected from the group consisting of (i) the group consisting of vasoactive agonists, vasopressor agents and vasoconstrictor drugs; (ii) an agent that acts on vasopressin receptors or adrenoreceptors; (iii) a calcium channel agonist; (iv) an agonist of the α₁ adrenergic receptor; (v) alfuzosin, doxazosin, epinephrine, methoxamine, naphazoline, norepinephrine, phenylephrine, prazosin, terazosin, tetrahydrozaline, tamsulosin; (vi) an agonist of the 5HT₁B_{A}D receptor; (vii) almotriptan, avitriptan, frovatriptan, oxidesumitriptan, rizatriptan, zolmitriptan; (viii) chlorpheniramine, ethylnorepinephrine, mephenterine, metaraminol, oxymetazoline, oxymetazoline, phenylpropanolamine, potassium chloride, pseudoephidrine, propylhexadrine; (ix) ephedrine, angiotensin and vasopressin; (x) tetrahydrozoline HCl 0.05%, naphazoline HCl 0.03%, oxymetazoline HCl 0.025%; (xi) a vasoconstrictor extract selected from the group including ephedra sinica (ma huang), polygonum bistorta (bistort root), hamamelis virginiana (witch hazel), hydrastis canadensis (goldenseal), lycopus virginicus (bugleweed), aspidosperma quebracho (quebracho bianco), cytisus scoparius (scotch broom), guava extract, ellagic acid, caffeine, peppermint extract, chamomile oil, and cypress; (xii) an agent that positively affects the McKenzie vasoconstrictor assay; (xiii) topical corticosteroids, hydrocortisone, cortisol, synthetic corticosteroids, betametasone, fluticasone, mometasone; (xiv) antagonists of the β₂ adrenergic receptor, such as butaxamine.

In some embodiments, the at least one vasoconstrictor is selected from the group consisting of methoxamine, methylnorepinephrine, oxymetazoline, phenylephrine, metaraminol, 4-(1-naphthalen-1-ylethyl)-1H-imidazole (4-NEMD), clonidine, guanfacine, guanabenz, guanoxabenz, guanethidine, xylazine, methyldopa, apraclonidine, brimonidine, detomidine, dexmedetomidine, lofexidine, romifidine, tizanidine, xylometazoline, amidephrine, amitraz, anisodamine, ergotamine, indanidine, medetomidine, mephentermine, midodrine, mivazerol, naphazoline, norfenefrine, octopamine, phenylpropanolamine, rilmenidine, synephrine, talipexole, tetrahydrozoline, xylometazoline, dobutamine, dopamine, denopamine, xamoterol, salbutamol, levosalbutamol, fenoterol, terbutaline, pirbuterol, procaterol, bitolterol, rimiterol, carbuterol, tulobuterol, reproterol, dopexamine, arformoterol, bambuterol, clenbuterol, formoterol, salmeterol, orciprenaline, metaproterenol, ritodrine, hexoprenaline, indacaterol, amibegron, solabegron, arbutamine, befunolol, isoxsuprine, nylidrin, oxyfedrine, prenalterol, ractopamine, bromoacetylalprenololmenthane, broxaterol, cimaterol, higenamine, mabuterol, methoxyphenamine, tretoquinol, zinterol isoprenaline, isoproterenol, epinephrine, norepinephrine, cirazoline, etilefrine, amphetamine, tyramine, ephedrine, pseudoephedrine, cocaine, allobarbital, amobarbital, aprobarbital, barbital, butobarbital, cyclobarbital, ethallobarbital, heptabarbital, hexobarbital, methohexital, pentobarbital, phenobarbital, proxibarbal, reposal, secobarbital, talbutal, thiopental, vinylbital, vinbarbital, brotizolam, cinolazepam, doxefazepam, estazolam, flunitrazepam, flurazepam, flutoprazepam, loprazolam, lormetazepam, nitrazepam, nimetazepam, midazolam, quazepam, temazepam, triazolam CL-218872, eszopiclone, indiplon, necopidem, pazinaclone, ROD-188, saripidem, suproclone, suriclone, SX-3228, U-89843A, U-90042, zaleplon, zolpidem, zopiclone, glutethimide, methyprylon, pyrithyldione afloqualone, cloroqualone, diproqualone, etaqualone, mebroqualone, mecloqualone, methaqualone, methylmethaqualone acebrochol, allopregnanolone, alphadolone, alphaxolone, ganaxolone, hydroxydione, minaxolone, Org 20599, tetrahydrodeoxycorticosterone, dexmedetomidine, lofexidine, medetomidine, romifidine, tizanidine, xylazine agomelatine, melatonin, ramelteon, doxylamine, hydroxyzine, diphenhydramine, bromodiphenhydramine, carbinoxamine, orphenadrine, niaprazine, phenyltoloxamine, propiomazine, pyrilamine, scopolamine, aceburic acid, gamma-amino-beta-hydroxybutyric acid (GABOB), gamma-hydroxybutyric acid (GHB), sodium oxybate, Xyrem®, gamma-butyrolactone (GBL), 1,4-butanediol, 3-chloropropanoic acid, acetylglycinamide chloral hydrate, chloral hydrate, chloralodol, dichloralphenazone, paraldehyde, petrichloral, centalun, ethchlorvynol, ethinamate, hexapropymate, methylpentynol, meprobamate, carisoprodol, tybamate, methocarbamol, 2-methyl-2-butanol, acecarbromal, apronal, bromisoval, carbromal, clomethiazole, embutramide, etomidate, gaboxadol, loreclezole, mephenoxalone, sulfonmethane, trichloroethanol, triclofos, valerian, valnoctamide and trazadone. In some embodiments, the at least one vasoconstrictor is selected from the group consisting of α₁ agonists and β₂ blockers. In some embodiments, the at least one vasoconstrictor is selected from the group consisting of epinephrine, noepinephrine, and pharmaceutically acceptable salts thereof.

In some embodiments, the instructions or label explain how to administer the at least one vasoconstrictor in a concentration effective to raise the patient's core body temperature by 1° C. within a period of 60 minutes. In some embodiments, the instructions or label explain how to administer the at least one vasoconstrictor by application at a concentration and/or to an amount of skin effective to raise the patient's core body temperature by 1° C. within a period of 60 minutes. In some embodiments, the instructions or label instruct the user to apply the at least one vasoconstrictor in conjunction with the dermal application of at least one of an antiseptic, antibiotic, an antimycotic or an antiviral compound. In some embodiments, the kit further comprises at least one of an antiseptic, antibiotic, an antimycotic or an antiviral compound. In some embodiments, the label or instructions explain how to dermally apply the at least one vasoconstrictor in conjunction with a penentration enhancer. In some embodiments, the kit further comprises a penetration enhancer. In some embodiments, the kit contains one vasoconstrictor. In some embodiments, the kit contains more than one vasoconstrictor. In some embodiments, the kit contains a mixture of vasoconstrictors.

### DETAILED DESCRIPTION

Embodiments of the invention will be better understood from the following detailed description, as well as with reference to the figures, in which:

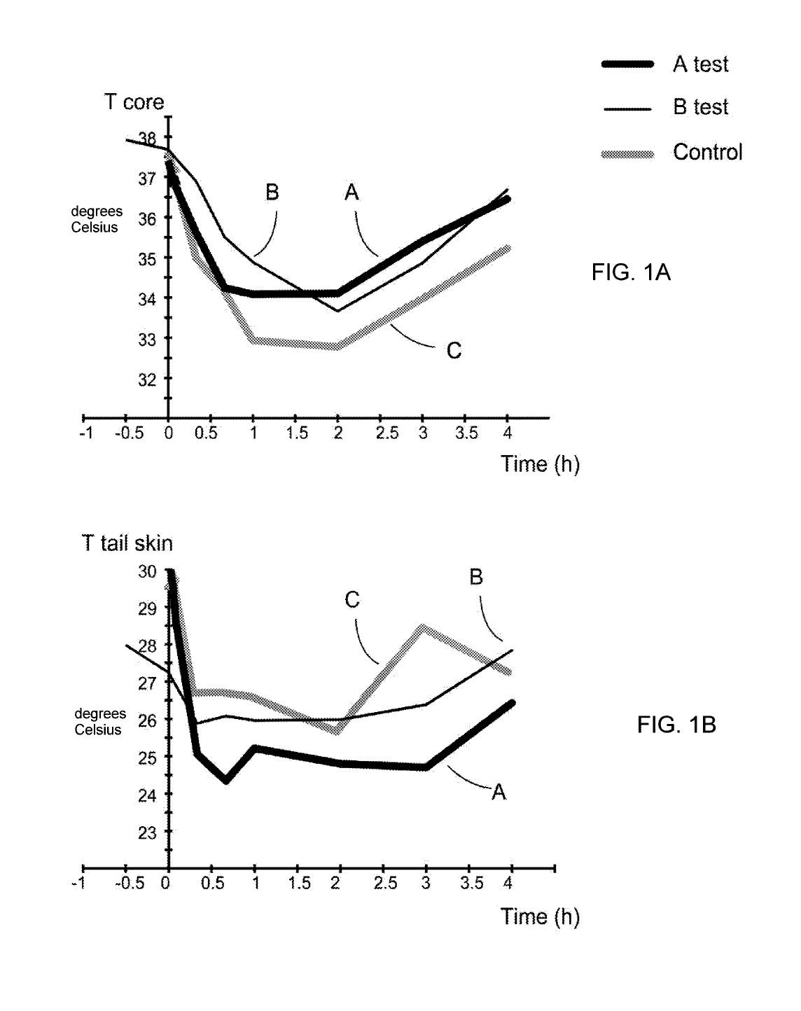

FIGS. 1A and 1B are graphs showing changes in mice in core body temperature and tail skin temperature, respectively, as a function of time;

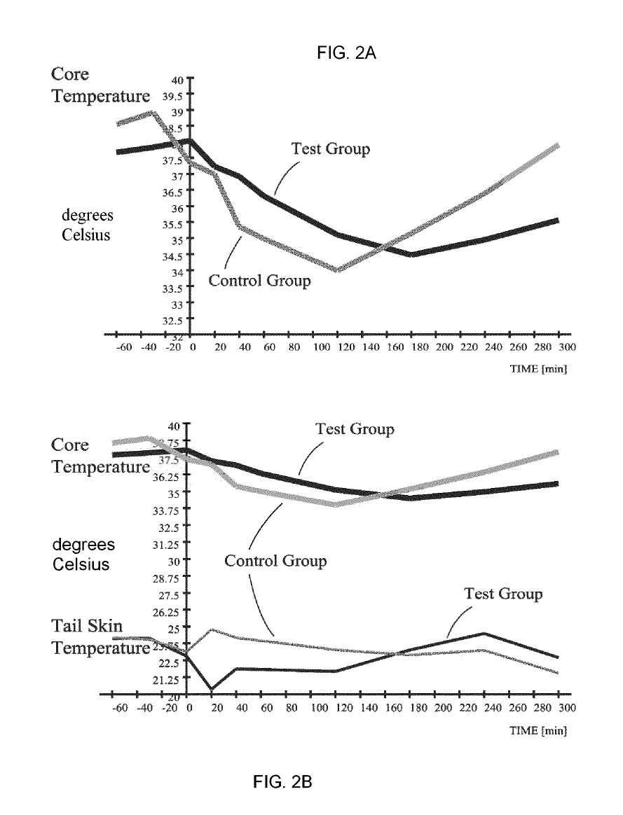

FIGS. 2A and 2B are graphs showing changes in mice in core body temperature and tail skin temperature, respectively, as a function of time; and

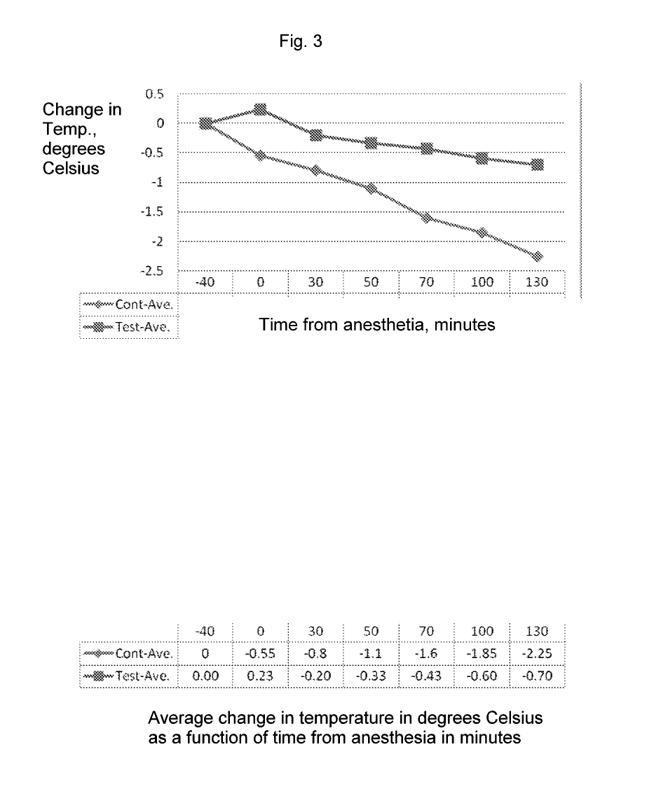

FIG. 3 shows changes in core body temperature in mice as a function of time.

In one aspect, there are provided in accordance with embodiments of the invention methods for treating, preventing or delaying the onset of anesthetic-induced hypothermia, by administering to a patient who is about to or who has already received general anesthetic a vasoconstrictor or mixture of vasoconstrictors. In some embodiments, the administering comprises applying the vasoconstrictor or mixture of vasoconstrictors to the skin of the patient. In another aspect, there are provided in accordance with embodiments of the invention methods for treating cancer by inducing hyperthemia in a patient suffering from cancer, in part by administering to a cancer patient a vasoconstrictor or mixture of vasoconstrictors, and in part by concomitantly administering a thermogenic substance and/or providing heat from an external source and/or administering MDMA. In some embodiments, the administering of the vasoconstrictor or mixture of vasoconstrictors comprises applying the vasoconstrictor or mixture of vasoconstrictors to the skin of the patient. In the context of this patent application, reference to “at least one vasocontrictor” will be understood as also encompassing a plurality of vasoconstrictors, including mixtures of vasoconstrictors, unless noted otherwise. Thus, for example, if it is stated that a dermally administrable composition containing the at least one vasoconstrictor is applied to at least 10% of a patient's skin, this includes the situation in which one composition comprising a first vasoconstrictor is applied to 5% of the patient's skin and a second composition comprising a second vasoconstrictor is applied to another 5% of the patient's skin. Furthermore, in cases where it is stated that the at least one vasoconstrictor is selected from a closed group of substances, it will be understood that if the at least one vasoconstrictor is utilized as one of a plurality of vasoconstrictors, separately or in a mixture, only one of the vacoconstrictors in the plurality need necessarily be selected from the members of the closed group.

Body thermoregulation involves three primary elements: heat production due to core body functioning, heat loss mostly through the skin surface organ, and regulatory signals from the hypothalamus brain organ. The different aspects and embodiments of the present invention use vasocontrictor substances to substantially reduce body heat loss to the environment through the skin. Use of additional supportive or enhancing elements in accordance with some embodiments, such as stimulation of core body functions and/or suppression of hypothalamus control and/or suppression of perspiration, are discussed herein in the context of specific applications and embodiments.

Vasoconstrictor substances per se—i.e. compounds that cause constriction of the blood vessels—are known in the art, but have not hitherto been used or suggested to be used in accordance with the different aspects of the invention described herein. For example, PCT patent publication WO 2006/138691 (hereinafter “PCT2006”) discloses pharmaceutical preparations containing vasoconstrictors and the use thereof to protect cells from the toxic side-effects of radiotherapy and cancer chemotherapeutic agents. Vasoconstrictor substances used in accordance with the teaching of PCT2006 are preferably agonists of the α₁ adrenergic receptor (preferred embodiments of which are stated to be epinephrine, phenylephrine, methoxamine, norepinephrine, tetrahydrozaline, naphazoline, prazosin, doxazosin, terazosin, alfuzosin, tamsulosin or any combination thereof) or agonists of the 5HT₁B_{A}D receptor (preferred embodiments of which are stated to be zolmitriptan, oxidesumitriptan, avitriptan, rizatriptan, almotriptan, frovatriptan, or any combination thereof). PCT2006 further teaches dosages of preferred embodiments.

U.S. Pat. No. 4,978,332 discusses local use of vasoconstrictors to inhibit the migration of cytotoxic drugs used for chemotherapy from the site of application of the cytotoxic drugs, by altering the blood flow serving the tumor/lesion area, so as to maintain the primary effect of the cytotoxic drug at the site of application.

Vasoconstrictive agents are described in Medical Pharmacology (1984), C. V. Mosby, Company, Chapter 15.

US Patent Publication Number 20030216364 teaches a topical dermatological composition with improved vasoconstrictor properties.

PCT patent publication WO 2004/032888 teaches a shaving cream composition containing a vasoconstrictor substance for preventing bleeding from nicks and cuts that occur while shaving the face, wherein the vasoconstrictor is phenylephrine, ephinephrine, norephinephrine, ethylnorepinephrine, potassium chloride, methoxamine, oxymetazoline), chlorpheniramine, phenylpropanolamine, tetrahydrozoline, pseudoephidrine, mephenterine, metaraminol, propylhexadrine, oxymetazoline, naphalozine, or a combination thereof, or a derivative of one of these compounds which functions as a vasocontrictor.

US Patent Publication Number 20070048234 teaches methods for treating acne using a vasoconstrictor in conjunction with an anti-acne agent, wherein said vasoconstrictor is selected from the group consisting of tetrahydrozoline HCl 0.05%, naphazoline HCl 0.03%, oxymetazoline HCl 0.025%, guava extract, ellagic acid, caffeine, cypress oil, witch hazel, peppermint extract, chamomile oil, and bugleweed.

PCT patent publication number WO 2007/023396 teaches a therapeutic kit to provide a safe and effective dosage of a vasoactive agent, including an aerosol packaging assembly,

wherein the vasoconstrictor is selected from the group consisting of: I. the group of vasoactive agonists, vasopressor agents and vasoconstrictor drugs; II. an agent that acts on vasopressin receptors or adrenoreceptors; III. a calcium channel agonist; IV. a vasoconstrictor selected from the group including ephedrine, epinephrine, phenylephrine, angiotensin and vasopressin; V. a vasoconstrictor selected from the group including ephedra sinica (ma huang), polygonum bistorta (bistort root), hamamelis virginiana (witch hazel), hydrastis canadensis (goldenseal), lycopus virginicus (bugleweed), aspidosperma quebracho (quebracho bianco), cytisus scoparius (scotch broom) and cypress; and VI. an agent that positively affects the McKenzie vasoconstrictor assay, and salts thereof.

PCT patent publication number WO 2006/031555 teaches cosmetically acceptable compositions which are particularly suited for skin lightening and for diminishing the appearance of ‘dark circles’ under the eyes. The compositions include any of several vasoconstrictors in a carrier with optionally added skin compatible ingredients.

Illustrative vasoconstrictive agents are: (1) sympathomimetics including the catecholamines, norepinephrine, epinephrine, isoproterenol, dopamine, and related compounds such as ephedrine and other phenylisopropylamines, phenylephrine, amphetamine, metraminol, methoxamine; (2) ergot alkaloids including lysergic acid, lysergic acid diethylamine, ergonovine, methylergonavine, methysergide, ergotamine; (3) the angiotensins; and (4) the prostaglandins. Vasoconstrictive agents are described in Medical Pharmacology (1984), C. V. Mosby, Company, Chapter 15.

The vasoconstrictor effect of topical corticosteroids, including its “skin blanching effect”, is discussed in the article by Smith et al. in Dermatology, 205 pp. 3-10 (2002). Topical corticosteroids include hydrocortisone, cortisol, and synthetic corticosteroids such as betamethasone, fluticasone and mometasone. They are applied to the skin in the form of creams, ointments and lotions and are the mainstay of controlling flare-ups of eczema. An advantage of corticosteroids in comparison to some other dermally-applied vasoconstrictors is in their multiple hours of effectiveness (about 10 hours).

Another group of compounds that act as vasoconstrictors are α-adrenoceptor agonists and antagonists. Initially classified as either α or β subtype receptors, based on anatomical location and functional considerations, more recent pharmacological and molecular biological techniques have identified the heterogeneity of the α-adrenoceptors and led to the identification of numerous subtypes of each receptor. α-Adrenoceptors exist on peripheral sympathetic nerve terminals and are divided into two subtypes, α₁ and α₂. α₁ is found mostly postsynaptically, while α₂, although typically sited presynaptically, can also occur postsynaptically. These initial subtypes were further divided into α₁ₐ, α_{1B} and α_{1D} receptors (by pharmacological methods), each with distinct sequences and tissue distributions, and α₁ₐ, α_{1b}, and α_{1d} by molecular biological and cloning techniques (note lower case letters refer to cloned receptors). Similarly, work done to identify subtypes of the α₂ adrenoeceptor has led to the discovery of a subclasses α_{2A}, α_{2B}, α_{2C} α_{2D}, and α_{2C10}.

α₁-adrenoceptors are found both in the central and peripheral nervous system. In the central nervous system they are found mostly postsynaptically and have an excitatory function. Peripherally, they are responsible for contraction and are situated on vascular and non-vascular smooth muscle. α₁-adrenoceptors on vascular smooth muscle are located intrasynaptically and function in response to neurotransmitter release. For non-vascular smooth muscle, they can be found on the liver, where they cause hepatic glycogenolysis and potassium release. On heart muscle they mediate a stimulatory (positive inotropic) effect. In the gastrointestinal system they cause relaxation of gastrointestinal smooth muscle and decrease salivary secretion.

The clinical uses of adrenergic compounds are vast. The treatment of many medical conditions can be attributed to the action of drugs acting on adrenergic receptors. For example, α-adrenoceptor ligands can be used in the treatment of hypertension. Drugs such as prazosin, an α₁ adrenoceptor antagonist and clonidine, an α₂-adrenoceptor agonist, both have antihypertensive effects. α₁ adrenoceptor antagonists are also employed in the treatment of benign prostatic hypertrophy.

TABLE 1

| Receptor Type | α₁ | A₂ |
|---|---|---|
| Selective Agonist | Phenylephrine, Methoxamine, | Clonidine, Clenbuterol |
| | Methylnorepinephrine, | |
| | Oxymetazoline | |
| Selective Antagonist | Doxazosin, Prazosin | Yohimbine, Idazoxan |
| Second Messengers | PLC activation via Gp/q Causes | decreases cAMP via Gi/o, causes |
| and Effectors | increases [Ca²⁺]ᵢ | decrease in [Ca²⁺]_{I} |
| Physiological Effect | Smooth muscle contraction | Inhibition of transmitter release, |
| | | Hypotension, anaesthesia, |
| | | Vasoconstriction |

Several sympathomimetic drugs are used primarily as vasoconstrictors for local application to nasal and ocular mucous membranes (see Table 1). α-adrenoceptor agonists are used extensively as nasal decongestants in patients with allergic or vasomotor rhinitis and in acute rhinitis in patients with upper respiratory infections (Empey et al., Drugs, June 1981, 21(6):438-443). These drugs probably decrease the resistance to airflow by decreasing the volume of the nasal mucosa. The receptors that mediate this effect appear to be the α₁-adrenoceptors, though α₂-adrenoceptors may be responsible for contraction of arterioles that supply the nasal mucosa. While a major limitation of therapy with nasal decongestants is that of a loss of efficacy with prolonged use, agonists that are selective for α₁ receptors may be less likely to induce mucosal damage (DeBernadis et al. 1987). As an ocular decongestant, to decrease swelling and redness of the eyes, α-adrenoceptor agonists are widely used in the treatment of allergic conjunctivitis, whether seasonal (hay fever) or perennial.

The following is a non-exhaustive list of agents that may function as vasoconstrictors.

α₁ adronergic receptor agonists: methoxamine, methylnorepinephrine, oxymetazoline, phenylephrine, metaraminol

α₂ adronergic receptor agonists: 4-(1-naphthalen-1-ylethyl)-1H-imidazole (4-NEMD), clonidine, guanfacine, guanabenz, guanoxabenz, guanethidine, xylazine, methyldopa, apraclonidine, brimonidine, detomidine, dexmedetomidine, lofexidine, romifidine, tizanidine, xylometazoline, amidephrine, amitraz, anisodamine, ergotamine, indanidine, medetomidine, mephentermine, midodrine, mivazerol, naphazoline, norfenefrine, octopamine, phenylpropanolamine, rilmenidine, synephrine, talipexole, tetrahydrozoline, xylometazoline, dexmedetomidine, lofexidine, romifidine, tizanidine, xylazine

β₁ adronergic receptor agonists: dobutamine, dopamine, denopamine, xamoterol

β₂ adronergic receptor short acting agonists: sulbutamol, levosalbutamol, fenoterol, terbutaline, pirbuterol, procaterol, bitolterol, rimiterol, carbuterol, tulobuterol, reproterol, dopexamine

β₂ adronergic receptor long acting agonists (LABA): arformoterol, bambuterol, clenbuterol, formoterol, salmeterol, orciprenaline, metaproterenol, ritodrine, hexoprenaline, indacaterol

β₃ adronergic receptor agonists: amibegron, solabegron, arbutamine, befunolol, isoxsuprine, nylidrin, oxyfedrine, prenalterol, ractopamine, bromoacetylalprenololmenthane, broxaterol, cimaterol, higenamine, mabuterol, methoxyphenamine, tretoquinol, zinterol

Nonselective β adronergic receptor agonists: isoprenaline, isoproterenol

Dual α/β adronergic receptor agonists: epinephrine {α₁₊₂, β₁₊₂}, norepinephrine {α₁₊₂, β₁}, cirazoline, etilefrine,

Indirect/mixed indirect presynaptic norepinephrine release: amphetamine, tyramine

Mixed: ephedrine, pseudoephedrine, cocaine, allobarbital, amobarbital, aprobarbital, barbital, butobarbital, cyclobarbital, ethallobarbital, heptabarbital, hexobarbital, methohexital, pentobarbital, phenobarbital, proxibarbal, reposal, secobarbital, talbutal, thiopental, vinylbital, vinbarbital; brotizolam, cinolazepam, doxefazepam, estazolam, flunitrazepam, flurazepam, flutoprazepam, loprazolam, lormetazepam, nitrazepam, nimetazepam, midazolam, quazepam, temazepam, triazolam; CL-218,872, eszopiclone, indiplon, necopidem, pazinaclone, ROD-188, saripidem, suproclone, suriclone, SX-3228, U-89843A, U-90042, zaleplon, zolpidem, zopiclone; glutethimide, methyprylon, pyrithyldione; afloqualone, cloroqualone, diproqualone, etaqualone, mebroqualone, mecloqualone, methaqualone, methylmethaqualone; acebrochol, allopregnanolone, alphadolone, alphaxolone, ganaxolone, hydroxydione, minaxolone, Org 20599, tetrahydrodeoxycorticosterone; melatonin, agomelatine, ramelteon; doxylamine, hydroxyzine, diphenhydramine, bromodiphenhydramine, carbinoxamine, orphenadrine, niaprazine, phenyltoloxamine, propiomazine, pyrilamine, scopolamine; aceburic acid, gamma-amino-beta-hydroxybutyric acid (GABOB), gamma-hydroxybutyric acid (GHB), sodium oxybate, Xyrem®, gamma-butyrolactone (GBL), 1,4-Butanediol, 3-Chloropropanoic acid; acetylglycinamide, chloral hydrate, chloralodol, dichloralphenazone, paraldehyde, petrichloral; centalun, ethchlorvynol, ethinamate, hexapropymate, methylpentynol; meprobamate, carisoprodol, tybamate, methocarbamol; 2-methyl-2-butanol, acecarbromal, apronal, bromides, bromisoval, carbromal, clomethiazole, embutramide, etomidate, gaboxadol, loreclezole, mephenoxalone, sulfonmethane, trichloroethanol, triclofos, valerian, valnoctamide, trazadone.

Other non-limiting examples of vasoconstricting agents contemplated for use in accordance with embodiments of the invention include tetrahydrozoline HCl 0.05%, naphazoline HCl 0.03%, oxymetazoline HCl 0.025%, guava extract, ellagic acid, caffeine, cypress oil, hamamelis (witch hazel), peppermint extract, chamomile oil, and bugleweed.

In some embodiments, the vasoconstrictor is selected from the group consisting of methoxamine, phenylephrine, 4-NEMD, clonidine, methyldopa, dobutamine, salbutamol, terbutaline, and isoprenaline.

The compound 3,4-methylenedioxymethamphetamine (MDMA), colloquially known as ecstasy, also bears mention, as hyperthermia is a known side effect of MDMA ingestion; hyperthermia due to MDMA can be so severe that a prime cause of death due to MDMA intake is ascribed to hyperthermia heat shock. Thus, oral or intravenous administration of MDMA or related compounds can be useful adjuncts to dermal application of a vasoconstrictor in inducing hyperthermia in cancer patients and achieving hyperthermia in accordance with embodiments the present invention.

The mechanism by which MDMA helps induce hyperthermia is not yet known. It has been speculated in the literature that the hyperthermia associated with MDMA intake is due in part due to its vasoconstrictor properties. For example the article by Blessing, J. Neurosci. 2003 July 16, 23(15):6385, states that “In rabbits, sympathetically mediated vasoconstriction in heat-exchanging cutaneous beds (ear pinnae) contributes to MDMA-elicited hyperthermia”. But there several other speculations as to the basis for the hyperthermia effect of MDMA, such as the drug's activating effect on serotonergic pathways, and no consensus opinion has been established. The conclusion of a recent review article (Easton and Marsden, J. Psychopharmacol, 2006, 20 (2), p. 194) is that “MDMA increases metabolic rate and evaporative water loss but at present we do not know much about what happens to cutaneous blood flow during MDMA-induced hyperthermia, either in humans or experimental animals”.

Furthermore, human clinical studies report that MDMA produces little or no hyperthermic response in humans at all doses tested (up to 2.0 mg/kg) under laboratory conditions (Liechti, Eur Neuropsychopharmacol. 2000; 10(4):289); this indicates that MDMA on its own is insufficient to consistently generate the level of hyperthermia needed for effective cancer treatment.

Malignant hyperthermia has more often been observed in people who have taken ecstasy analogues such as para-methoxyamphetamine (PMA) or 4-methylthioamphetamine (4-MTA). Malignant hyperthermia usually occurs as a secondary consequence of serotonin syndrome, a condition in which too much serotonin is released into the brain. This can occur with MDMA if too much 5-hydroxytryptophan (5-HTP) is consumed alongside MDMA, as this can overload the brain with serotonin once the 5-HTP gets converted to serotonin. As a guideline, 50-200 mg of 5-HTP will make MDMA work better and last longer, but with more than 300 mg 5-HTP, the user can start to become confused, red-faced, hot and dry to the touch; this is serotonin syndrome, which can lead into lethal malignant hyperthermia if it becomes too severe.

It is known that the hyperthermic effects of MDMA vary with external ambient temperature. A study examined the effects of ambient temperature on core body temperature in recreational users of MDMA; however, unlike findings in rats, MDMA produced hyperthermia regardless of ambient temperature (Freedman et al., Psychopharmacology (2005) 183:248-56), suggesting heat loss impairment. However, due to ethical constraints this study was limited to a single exposure of MDMA at a dose which is lower than that ingested by the average MDMA user. Recent experiment in rhesus monkeys showed no significant dependence of the hyperthermic effect of MDMA on ambient temperature up to 31° C. In particular, the hyperthermic rise was not significantly more than 1° C. compared to the control (Banks et al., Drug Metabolism and Disposition 35:1840-1845 (2007)].

Altogether, the hyperthermic effect of MDMA in human subjects remains unsystematic in reproduction and unclear as to its underlying causes. Peak plasma concentrations of MDMA are nonlinear with respect to dosage, becoming disproportionately elevated with larger doses. Peak plasma concentrations of a moderate recreational dose of MDMA (1.5 mg/kg) reached an average of 0.331 ng/ml at about two hours after oral administration in humans. Experiments in monkeys indicate a flat peak of thermoregulatory disruption effect is reached about 90 minutes after intravenous injection and lasting for about 3 hours. To the extent that a similar phenomenon is exhibited in humans, it would be useful to coordinate the effects of the vasoconstrictor(s) with the occurrence of the flat peak of MDMA thermoregulatory effects.

Furthermore, other stimulant drugs such as methamphetamine and cocaine are also known cause an increase in body temperature, although it is unclear if the mechanism is similar to that for MDMA. Thus, oral or intravenous administration of MDMA or related compounds can be useful adjuncts to dermal application of a vasoconstrictor in inducing hyperthermia in cancer patients.

Skin Penetration Enhancers. In some embodiments of the invention, a penetration enhancer is used to facilitate the penetration of vasoconstrictor into the skin. Some techniques for improving the delivery of drugs through the skin are discussed in Benson, Current Drug Delivery, 2005, 2:23-33. Suitable skin penetration enhancers include, for example, surfactants such as sodium laurate, sodium lauryl sulfate, cetyltrimethylammonium bromide, benzalkonium chloride, Poloxamer (231, 182, 184), Tween (20, 40, 60, 80) and lecithin (U.S. Pat. No. 4,783,450); the 1-substituted azacycloheptan-2-ones, particularly 1-n-dodecylcyclazacycloheptan-2-one; alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate; amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; organic acids, particularly salicylic acid and salicylates, citric acid and succinic acid.

Generally, vasoconstrictors in accordance with embodiments of this invention may be dissolved in an appropriate pharmaceutically or cosmetically acceptable solvent and applied directly to the skin. In some embodiments, the vasoconstrictors are combined with a cream or gel base, so that application of the vasoconstrictor is more easily localized and controlled. Most pharmaceutically or cosmetically acceptable gel or cream bases may be used. Suitable gels include, for example, cellulose-based gels, (e.g., hydroxyethyl cellulose (HEC), hydroxypropyl cellulose (HPC), and carboxymethyl cellulose (CMC)) and acrylate copolymers. Suitable cream bases include emulsions formed from a water phase of a humectant, a viscosity stabilizer and water, an oil phase of fatty acid alcohol, a semi-solid petroleum hydrocarbon and an emulsifying agent and a phase containing the vasoconstrictor of the invention dispersed in an aqueous stabilizer-buffer solution. If desired, stabilizers, such as are known in the art or may be developed later, may be added in accordance with embodiments of the invention. Cream-based pharmaceutical formulations containing the vasoconstrictor may be contain, for example, aqueous emulsions containing a fatty acid alcohol, semi-solid petroleum hydrocarbon, 1,2-ethyleneglycol and an emulsifying agent.

In some embodiments, administration of a vasoconstrictor substance to cancer patient, for example application of a vasoconstrictor substance to the skin of a cancer patient, is done in conjunction with administration of another substance that will help raise body temperature; in some embodiments that substance is a thermogenic substance. In the context of this patent application, a “thermogenic substance” refers to a substance which increases the metabolism of the body, thereby generating heat. Common natural thermogenic substances are ephedra, bitter orange, capsicum, ginger and caffeine. The ECA Stack (ephedrine, caffeine, aspirin) is the most well-known thermogenic formulation, popular among bodybuilders as a means to achieve low body fat levels.

On technique known and used to raise the Basal Metabolic Rate (BMR) is exposing the body to very cold temperatures, usually by soaking the body in icy cold water until a person can not tolerate it any longer, almost to the point of inducing hypothermia. This causes a release of thyroxine from the thyroid gland by means of the sympathetic nervous system, thus raising BMR, resulting in an overall increase in body temperature.

Thus stimulating the thyroid gland to release thyroxine is a general method of raising BMR, resulting in an overall increase in body temperature. Thyroxine (T4) and triiodothyronine (T3) are substances release by the thyroid gland. T3 is an active form of thyroid hormone. T3 in turn is believed to increase the efficiency with which the human body burns calories, thereby generating heat. Hence, thyrotropic (thyroid gland stimulators) and/or T3 donor substances are thermogenic substances as that term is used in the present application. One can also directly administer synthetic T3 or T4 (Synthroid).

Thyrotropic thyroid stimulation hormone (TSH) has been described in the literature, as has a test for thyroid releasing factor (TRF), which acts to release TSH. For example, Yamazaki in J. Clin. Endocrin. & Met., 80:473 (1995) reported that bovine TSH (bTSH) was approximately 6- to 9-fold more active than human TSH (hTSH).

Stimulators of T4 and T3 release include (1) TSH, which is the primary stimulator; (2) vasoactive intestinal peptide (VIP) and β-adrenergic agonists, both of which act through the adenylcyclase-cAMP pathway; (3) Long-Acting Thyroid Stimulators (LATS) are Antibodies (Ab) which mimic the actions of TSH; these also sometimes called Thyroid Stimulating Immunoglobulins. Because LATS are not down-regulated by high blood levels of thyroid hormones, their expression at high levels represent a pathological state.

Substances which block α₂ adrenoreceptors, in addition to functioning as vasoconstrictors, may in some cases also act as thermogenic substances. For example, yohimbine works by blocking α₂ adrenoreceptors. There are a number of feedback mechanisms that prevent the release of norepinephrine (NE), one of the body's primary lipolytic hormones. When NE is released, such as in periods of stress or after taking a sympathomimetic (such as ephedrine), it stimulates both the α and β adrenoreceptors. Stimulation of the beta adrenoreceptors causes the breakdown of fat while stimulating the α₂ adrenoreceptors has the opposite effect, preventing the release of NE and lipolysis. Yohimbine prevents this negative feedback mechanism, thus increasing NE release and lipolysis. There are reasons why α₂ inhibition is specifically useful. While the β-adrenergic system primarily controls lipolysis during periods of intense activity, during rest, which makes up most of our day, the α-adrenergic system is in control. The increase of lipolysis is tantamount to thermogenesis. Therefore, α₂ blockers in general and Yohimbine in particular, are considered thermogenic substances in the present application.

Some substances may be thermogenic substances in one context but vasoconstrictors in another context. For example, caffeine, when ingested orally and absorbed into the core body blood stream, has the effect of increasing body metabolism, thereby being thermogenic, and dilating the core blood vessels. However, when applied dermally, caffeine has the effect of constricting cutaneous blood vessels.

Another dual action substance is ephedrine, an alkaloid from the leaves of Ephedra equisetina, E. sinica, and other species (family Gnetaceae), or produced synthetically; commonly used salts are ephedrine hydrochloride and ephedrine sulfate. Ephedrine is an adrenergic (sympathomimetic) agent, an alpha- and beta-adrenergic agonist, that may also enhance release of norepinephrine. Its actions similar to those of epinephrine. When inhaled, it is used as a bronchodilator, mydriatic, or pressor agent. When used topically on skin, it is a vasoconstrictor.

Circulation through the skin serves two major functions: nutrition of the skin tissue and heat transfer. These two functions have different hemodynamic requirements. Nutrient exchange requires slow movement of blood through thin-walled, small diameter vessels with walls permeable to small molecules. Heat exchange requires the movement of large volumes of blood through vessels closely at or near a body surface. Accordingly, two types of vascular structures are found in the skin of mammals: 1) nutritive units consisting of arterioles, capillaries, and venules, and 2) heat-transfer units consisting of venous plexuses (dense networks of thick-walled, large-diameter venules) and arteriovenous anastomoses (AVAs; vascular communications between small arteries and the venous plexuses).

The heat-transfer vascular units and nutrient vascular units are anatomically distinct and have mutually exclusive functions. Nutritive vascular units are uniformly distributed throughout the skin, whereas the heat exchange units are found only in non-insulated skin regions; in humans these are the palms of the hands, the soles of the feet, the ears, and non-hairy regions of the face (Bergersen, 1993; Gemmell and Hales, 1977; Saad, 2001). Heat exchange units also exist in the footpads and tongues of dogs (Baker, 1982), ears of elephants (Phillips and Heath, 1992) and rabbits (Ootsuka et al., 2003), and tails of rodents (Heath, 1998, Johnson 2002). In the human hand, AVAs and associated venous plexuses are found under the nail beds, the tips of the digits, the palm, and the palmar surface of the fingers. AVAs and venous plexuses are absent from the dorsal surface of the fingers and hand (Roddie, 1983). The dimensions of venous plexuses determine the blood volume capacity of a heat exchange region while the AVAs control the blood flow through the venous plexuses. The heat exchange-vascular units do not contribute to the nutrition of surrounding tissues, and the nutritive units are not directly active in temperature regulation. The thermoregulatory vascular units enable direct heat transfer from the body core to the surrounding environment (Krauchi et al., 1999, 2000). Blood passing through these heat exchange units is delivered directly from the heart via the arterial system and is delivered back to the heart via venous return. Blood flow through the heat exchange vascular units is extremely variable. It has been estimated that blood flow into the venous plexuses can range from near zero in cold stress to as much as 60% of the total cardiac output during heat stress (Greenfield, 1983; Johnson and Proppe, 1996). Constriction of the AVAs helps to thermally isolate the body core from the environment. Conversely, dilation of the AVAs promotes a free exchange of heat between the body core and the environment. Consequently, in accordance with some embodiments of the invention, the vasoconstrictor substance is an AVA constrictor.

Studies have shown that several 5-HT_{1B/1D} receptor agonists potently constrict porcine carotid arteriovenous anastomoses, and it is likely that the same is true for humans. Such agonists include sumatriptan and alniditan (De Vries et al., Eur. J. Pharmacol. 1998 351:193-201). In another study, it was found that adrenergic α₁-stimulation with phenylephrine produced AVA constriction. Adrenergic α₂-stimulation with UK-14304 (5-bromo-N-(4,5-dihydro-1H-imidazol-2-yl)-6-quinoxalinamine) produced constriction of the AVA, norepinephrine (a mixed α₁- and α₂-agonist) also produced AVA vasoconstriction. Overall, AVAs contain a heterogeneous mixture of both α₁- and α₂-receptors, and α₂-receptors may have a greater influence than α₁-receptors on overall tone in AVA (Pollock et al., Am. J. Physiology, Heart and Circulatory Physiology, 1996, 40:5, pp. H2007).

In accordance with some embodiments, the one or more vasoconstrictor substances are administered topically to the skin. This method of administration decreases systemic effects of the vasoconstrictors, limiting the constriction of interal blood vessels and concentrating the vasoconstriction near the area of application.

To be effective as a stand-alone, full body thermoregulating method, dermal administration of vasoconstrictor substances needs to be conducted over a substantial portion of the skin surface of the human body. Hence, an associated kit may contain (i) sufficient quantities of vasoconstrictor substance in a form appropriate for topical application, such as a gel, cream, foam, or ointment (or powder suitable for mixture within a liquid), and/or (ii) appropriate instructions.

Inducing therapeutic hyperthermia. As explained in Hyperthermia 2006, the body temperature is set by the steady state flow of heat from inside the body to the outside via the skin boundary. It is mostly the blood flow which carries transport this heat. Pyrogens influence the brain thermostat regulating the skin blood vessels dilatation. External devices either mechanically impede skin heat loss or even further heat the skin surface. In accordance with one aspect of the present invention, cutaneous application of one or more vasoconstrictor substances is used in conjunction with one or more thermogenic substances and/or an external source of heat for inducing therapeutic hyperthermia in cancer patients, as a method for treating the cancer. The use of the vasocontrictor substances reduces body heat loss to the environment, and thereby helping to induce hyperthermia for effective treatment of cancer patients.

Preferably, therapeutic hyperthermia treatments for cancer show the ability to sustain the following protocols of individual treatment sessions: (i) 1-2 h duration at 41-42° C., or (ii) 3-8 h at 39-40° C. In the context of the present application, the statement “desired therapeutic hyperthermia level” should be understood as referring to a core body temperature above 38° C.

In those embodiments that involve inducing hyperthermia, the use of a vasoconstrictor may be accompanied (simultaneously or sequentially) by administration of one or more thermogenic substances, which increase core body heat production. The combination of a thermogenic substance with a vasoconstrictor can enable the use of lower dose of vasoconstrictor than would be required to reach the desired hyperthermia level than if the vasoconstrictor was used alone. In some embodiments, the combined use of the thermogenic substance with the vasoconstrictor yields synergistic effects. Since the response of individual patients to particular vasoconstrictor and/or thermogenic substances may vary, the combined use of these two types of substances gives two degrees of freedom to tailor the respective dosages to the individual patient as desired for a particular level of hyperthermia treatment.

The hyperthermia level achieved through the use of vasoconstrictors is also dependent on the external ambient temperature. Hence, in some embodiments of the present invention, the hyperthermia temperature level can also be tuned, in part, by controlling the room temperature in which the patient resides during treatment, or by application of heat from a different source external to the patient.

Peak plasma concentrations of orally-administered MDMA are nonlinear to dosage, becoming disproportionately elevated with larger doses. Peak plasma concentrations of a moderate recreational dose of MDMA (1.5 mg/kg) reached an average of 0.331 ng/ml at about two hours after oral administration in humans. Thus in some embodiments, dosages higher than 1.5 mg/kg are administered in conjunction with a topical vasoconstrictor in order to consistently effectuate therapeutic levels of hyperthermia.

The hypothalamus sends regulating signals in an attempt to bring body temperature into a normal temperature range. In order to achieve a stable state of hyperthermia, it may be advantageous to suppress the thermoregulatory functioning of the hypothalamus. Substances which act to such effect will be referred to as “hypothalamus-thermoregulation suppressors” in the context of the present application. For example, MDMA may act as a hypothalamus-thermoregulation suppressor. Some anesthetics also act as hypothalamus-thermoregulation suppressors. Hence, in some embodiments, a hyperthermia treatment utilizes the combination of topical application of a vasoconstrictor agent, anesthesia, and an ambient temperature of over 30° C.

It is known that the vasoconstriction effect of various substances may vary substantially from person to person, and hence the strength of the hyperthermic effect in a given individual of dermal application of vasocontrictor, together with administration of a thermogenic substance and/or heating cannot be fully anticipated. There is a risk for it to cross the prescribed safety limits for duration and strength of hyperthermia treatment. Prescribed guidelines of hyperthermia treatment are exemplified in the tables listed in the quoted Hyperthermia 2006 article. Thus, in some embodiments, in order to avoid hyperthermia treatment continuing for a duration or reaching a temperature that is unsafe and may cause injury, safety measures for fast vasodilatation to end the hyperthermic state are present when the method is being practiced. In some cases, the treatment kit in which the vasoconstrictor and/or thermogenic substance is provided may also contain a vasodilator drug or other means to terminate the hyperthermic state. In some embodiments, vasodilators are used in delayed release forms to guarantee the ending of hyperthermia treatment at a prescribed time. Thus, in some embodiments; there are provided vasodilators for use as a hyperthermic relief element in a kit for hyperthermia treatment of cancer patients, and instructions for use of the vasodilator in accordance with the measure and duration of the induced hyperthermia condition. In some embodiments, the vasodilator is integrated for delivery within a combination composition that also comprises the vasoconstrictor.

An example of such a vasodilator is vitamin B3 (niacin). Elaboration of optional vasodilator substances is provided in PCT application documents PCT/112008/000508 title “Anti-Pyretic Vasodilators”, which is hereby incorporated in its entirety. Other examples of such vasodilator are nitric-oxide donor substances, such as nitroglycerin.

While the initial use of hyperthermia treatment in accordance with embodiments of the present invention should be conducted under close medical supervision, once the characteristic response of an individual patient to the treatment is established, the patient may continue the treatment at his own convenience.

Prevention and amelioration of hypothermia due to general anesthesia. Hypothermia is defined as body temperature below 36° C. Hence, in the context of the present invention, treating hypothermia should be understood as raising core body temperature to or above 36° C., and preventing hypothermia should be understood as maintaining core body temperature at or above 36° C.

With regard to post-operative hypothermia induced by anesthetic, in accordance with embodiments of the present invention, application of vasoconstrictor substances to the skin of patients can be used to induce vasoconstriction in the skin to prevent or ameliorate hypothermia. Application of the vasoconstrictor make take place shortly prior to start of surgery, e.g. within 30-60 minutes of the commencement of surgery, thereby providing prophylaxis of hypothermia; or it may conducted during or after surgery to mitigate or ameliorate the hypothermia condition. Topical application of the vasoconstrictor to the skin concentrates the vasoconstrictor effects in the skin, and minimizes systemic effects of the vasoconstrictor. Optionally, application of one or more vasoconstrictors to the skin may be accompanied by application of heat from a source external to the patient.

As discussed in PCT2006, several applications of a vasoconstrictor may be needed in order to induce and maintain a high degree vasoconstriction (evidenced in visible skin blanching) for extended periods of time.

Synergetic combination formulas: In some embodiments, a combination of vasoconstrictors is used in order to obtain extended elevated vasoconstrictor effects. For example, some topical vasoconstrictors, such as epinephrine, initially act quickly (within about 20 minutes of application), but their activity diminishes significantly after 3 hours, whereas topical corticosteroids only begin to have significant vasoconstrictor effect about 7 hours after application, with lasting effects up to about 17 hours after application. Other vasoconstrictors, such as phenylephrine, when applied dermally, show intermediate periods of activity when applied topically, beginning to show their vasoconstricting effects after about 3 hours after application.

Therefore, in accordance with embodiments of the invention, the use of different combinations of vasoconstrictors, applied separately or in single formulation, can be conceived to address need of treatment for different lengths of time. For example, prevention or amelioration or minimization of hypothermia according to embodiments of the present invention can be fitted to different time scales depending on the planned duration of anesthesia associated with different known surgical operations.

Furthermore, in the context of inducing hyperthermia, the use of vasoconstrictor substances alone increases core body temperature; nevertheless, in order to achieve desired therapeutic levels of hyperthermia, it may be desired to use vasoconstrictors in conjunction with a thermogenic agent and/or an application of heat from an external source, particularly if application of the vasoconstrictor alone will not be sufficient, or the required dosage of vasoconstrictors will be excessively high.

The combined application of vasoconstrictor and thermogenic substances is not linearly additive in the following sense: if dosage D_{V} of vasoconstrictor V causes a temperature rise of Tᵥ degrees in a given patient, and a dosage D_{TH} of thermogenic substance TH causes a temperature rise of Tₜₕ degrees in same given patient, then the combined application of D_{V}+D_{TH} causes a rise of temperature T_{c} which is not equal to Tᵥ+Tₜₕ. The non-linear dependence of T_{c} on (Tᵥ, Tₜₕ) is a statement of the non-trivial synergistic effect of the combined application of vasoconstrictor and thermogenic substances. In particular, from a therapeutic perspective, in some embodiments, it will be appreciated that for dosages D_{V} and D_{TH}, neither the corresponding Tᵥ nor Tₜₕ alone is in the desired therapeutic hyperthermia level range, yet the combination D_{V}+D_{TH} gives rise to T_{c} in the desired therapeutic hyperthermia level range.

Diosmin prolongs the vasoconstrictor effect of noradrenaline on the vein wall, increasing venous tone, and therefore reducing venous capacitance, distensibility, and stasis. Hence, a combination of vasoconstrictor agent and diosmin is provided in accordance with some embodiments of the invention.

As used herein, and as would be understood by the person of skill in the art, the recitation of a name of a compound is intended to include salts, solvates and inclusion complexes of that compound. Thus, in accordance with some embodiments of the invention, a compound as described herein, including in the contexts of pharmaceutical compositions, methods of treatment, and compounds per se, is provided as the salt form. Furthermore, when reference is made in a claim to a compound or a pharmaceutically acceptable salt thereof, it will be understood that claims which depend from that claim which refer to such a compound also include pharmaceutically acceptable salts of the compound, even if explicit reference is not made to the salts in the dependent claim.

Stimulating lipolysis. Lipolysis, the breakdown of lipids in the body, is a process which releases heat. Therefore, stimulation of lipolisys increases total heat production in the body, and contributes to elevating body temperature. Hormones which when injected or ingested orally are known to induce or stimulate lipolysis include epinephrine, norepinephrine, glucagon and adrenocorticotropic hormone. In the context of the present application, lipolysis stimulators are included under the rubric of thermogenic substances. In addition, α₂ adrenoreceptor blockers can act to effectively increase lipolysis. Stimulation of the β adrenoreceptors causes the breakdown of fat while stimulating the α₂ adrenoreceptors has the opposite effect, preventing the release of norepinephrine (NE) and lipolysis. α₂ blockers prevent this negative feedback mechanism, thus increasing NE release and lipolysis.

Preventing associated high blood pressure. It will be appreciated that constriction of skin capillaries, e.g. as a result of topical vasoconstrictor application, may also result in undesired high blood pressure. In order to counteract this effect, in some embodiments of the present invention internal application (oral or intravenous) of a vasodilator is included. The vasodilator is preferably of a nitric-oxide donor type (e.g., nitroglycerine and derivatives) which primarily dilate internal, large blood vessels, and has relatively marginal effect on skin capillaries.

To minimize the potential influence of dermal application of vasoconstrictor on central blood pressure and heart rate, in some embodiments of the present invention the one or more vasoconstrictors are selective alpha agonist substances, such as phenylephrine or methoxamine.

Antiseptic combination. In the context of surgical operations, there is value to have the patient body skin surface as sterile as possible. Disinfection by topical alcohol swabs is common in patient preparation for surgery. Hence, in some embodiments of the present invention, the composition containing one or more vasoconstrictors further comprises a disinfecting agent, as known in the art.

Topical Administration. As stated, in some embodiments, topical dermal administration of vasoconstrictors is utilized; this minimizes systemic effects of the vasoconstrictors. Absorption through body outer surface skin is known to be less than via mucous membranes, e.g. via rectal, nasal or eye tissues. Hence, to achieve effective bioavailability of active vasoconstrictors, active vasoconstrictor concentration in the topical formulations will often need to be higher than 1%. The inclusion of a transdermal carrier and/or a skin penetration enhancer in such formulations may enable the concentration of the vasoconstrictors in the formulations to be reduced. Transdermal carriers and penetration enhancers known to those skilled in the art include polymethacrylic acid (PMA), carbopol, polyethylene glycol 8000 (PEG), propylene glycol (PG), water, alcohol, acetone, caprylic acid, caproic acid, oleic acid, lauric acid, isopropyl myristate, triethanolamine, or mixtures thereof. Transdermal penetration enhancers are also described, for example, in Karande et al., PNAS USA 102:4688-4693 (Mar. 29, 2005). However, some of these may not be suitable for use on patients that have very sensitive skin or allergies. In some instances it may be desirable to include non-proteinaceous carriers, so as to form a liquid, particularly an aqueous liquid, or semi-solid or gel medium. Substances which may find use are physiologically acceptable substances, such as carbohydrates, polylactate, agaroses, dextrans, cellulose, gums, etc. Synthetic peptides may find use, such as polylysine, polyarginine, etc. The composition may be formulated with lipids to form liposomes or in a solid form in combination with silicones, epoxide resins, hydroxyapatite, etc. The drugs and carrier will be selected to minimize any inactivating effects on the drugs.

The dermally administrable compositions used in accordance with embodiments of the invention include known pharmaceutical forms utilized for topical cutaneous administration, including solutions, gels, lotions, creams, ointments, foams, mousses, emulsions, microemulsions, milks, serums, aerosols, sprays, dispersions, microcapsules, vesicles and microparticles thereof. These compositions may be formulated according to techniques known in the art, or in accordance with techniques developed in the future.

The terms “pharmacologically acceptable carrier” and “dermatologically acceptable carrier”, as used herein, mean respectively that the carrier is suitable for administration to a person, is compatible with the active agents any other components present in the pharmaceutical composition, and will not cause any untoward safety or toxicity concerns; and that the carrier is suitable for cutaneous topical application, is compatible with the active agents any other components present in the pharmaceutical composition, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicon emulsions, are useful herein. As will be understood by the skilled artisan, a given component will distribute primarily into either the water or oil/silicon phase, depending on the water solubility/dispersibility of the component in the composition. A safe and effective amount of carrier is from about 50% to about 99.99%.

The composition, if desired, can contain excipients, binders, lubricants, disintegrants and the like, as is known in the art. If desired, it can also contain oily materials such as various fats, oils, waxes, hydrocarbons, fatty acids, higher alcohols, ester oils, metallic soaps, animal or vegetable extracts, hydrophilic or lipophilic gelling agents, hydrophilic or lipophilic active agents; additional pharmaceutically effective components such as vitamins, hormones, amino acids; surfactants, colorants, dyes, pigments, fragrances, odor absorbers; antiseptics, preservatives, bactericides; humectants, thickeners, solvents, fillers; antioxidants; sequestering agents; sunscreens; or other known components and additives that do not unduly impair the vasoconstrictive effects of the vasoconstrictor. Additional descriptions of suitable compositions for topical dermal administration, and components suitable for inclusion therein, are described in Remington's Pharmaceutical Sciences, 18th Edition, Mack Publishing Co., 1990, and Remington: The Science and Practice of Pharmacy, 20th Edition, edited by A. Genaro, Lippincott Williams and Wilkins, Baltimore, Md., 2000.

Examples of suitable oils includes mineral oils, plant oils such as peanut oil, sesame oil, soybean oil, safflower oil, sunflower oil, animal oils such as lanolin or perhydrosqualene, synthetic oils such as purcellin oil, silicone oils such as cyclomethicome among others. Fatty alcohols, fatty acids such as stearic acid and waxes such as paraffin wax, carnauba wax or beeswax may also be used as fats.

The composition may also contain emulsifying agents such as glyceryl stearate, solvents such as lower alcohols including ethanol, isopropanol, and propylene glycol, hydrophilic gelling agents including carboxyvinyl polymers or acrylic copolymers, polyacrylamides, polysaccharides, lipophilic gelling agents or fatty acid metal salts among others, hydrophilic acting agents such as amino acids, sugars, starch or urea, lipophilic active agents such as retinol or tocopherol.

In addition, the drug(s) can be employed encapsulated in liposomes or other controlled rate release compositions so as to provide for separate and distinct rates of release of the drug(s). Alternatively, other methods of encapsulation can be employed where one or more of the active ingredients are encapsulated in a biodegradable substance, where the rate of release is related to the thickness of the biodegradable coat.

The active ingredients used in accordance with embodiments of this invention may be uniformly dispersed in a physiologically acceptable medium, particularly aqueous, such as saline, phosphate buffered saline, distilled water, etc. The aqueous medium will be sufficient to provide for an amorphous dispersion, usually a solution, capable of flowing under mild pressure.

In addition to the vasoconstrictor(s) and optional thermogenics, a number of minor components may also be included for a variety of purposes. These agents will for the most part impart properties which protect the stability of the composition, control the pH, or the like. Illustrative agents include phosphate or acetate buffers, methyl or propyl paraben, polyethylene glycols, etc. These agents generally will be present in less than about 2 weight percent of the total composition, usually less than about 1 weight percent, and individually may vary from about 0.001 weight percent to about 1 weight percent.

In preparing the pharmaceutical formulations, other materials, as appropriate, may be added concomitantly or sequentially. After ensuring the uniform dispersion of the various components in the mixture, the mixture may be sterilized and sealed in an appropriate container. In the event the various components are unstable or form undesirable complexes when stored in a mixture prior to administration, each component may be dispensed at an appropriate concentration into a separate container for mixing just prior to administration. Those components which are stable together may be dispensed together into a single container for mixture with one or more reagents containing those additional ingredients found to promote instability or to form undesirable complexes. A device or kit containing separate components may be prepared which facilitates easy formulation prior to administration. The concentration of each separate component is formulated so that the therapeutically effective concentration of each agent is achieved when all the separate components in the kit are admixed.

Combination with suppressing of hypothalamus thermoregulation. As mentioned above, to prevent resistance reaction of the body to desired hyperthermia induction, it may be desired in some embodiments to suppress thermoregulation by the hypothalamus, via application of a hypothalamus-thermoregulation suppressor substance. MDMA is one substance that is known to suppress thermoregulation by the hypothalamus; other selective serotonin reuptake inhibitors (SSRIs) may also serve the function of hypothalamus thermoregulation suppressors. In some embodiments, a hypothalamus thermoregulation suppressor is administered internally before the vasoconstrictor is administered topically; in other embodiments, a hypothalamus thermoregulation suppressor is administered internally after the vasoconstrictor has been applied topically but while the vasoconstrictor composition remains on the skin.

Combination with suppression of perspiration. At elevated temperatures, such as those desired for therapeutic hyperthermia, the human body normally reacts with increased perspiration. Thus, it may be desired to suppress perspiration in order to achieve and/or maintain hyperthermia. In some embodiments of the invention, the composition for topical administration further comprises of an antiperspirant substance; in some other embodiments, a topical antiperspirant may be administered separately. Furthermore, the utility of perspiration is greatly reduced with increased ambient humidity; effectiveness of perspiration is essentially eliminated with an ambient humidity of above 75%. In some embodiments of the present invention for the purpose of inducing therapeutic hyperthermia to treat cancer, the patient is maintained in an ambient humidity of at least 50%, in some embodiments at least 60%, 70%, 75%, or 80% depending on individual properties and combination with other elements of the treatment regimen (e.g., ambient temperature).

Combination with glove/sock peripheral warming devices. The use of external warming devices for selectively warming the palms and/or the feet of human subjects is particularly effective for warming of core body. Hence, in some embodiments, a glove shaped and/or sock shaped warming pad, optionally formed to be fitted over human hands and/or feet, respectively, may be utilized in conjunction with the dermal application of one or more vasoconstrictors. Such pads may contain heated gels or liquids or heat radiation sources as know in the art (e.g. warming pads, warming covers, warming bottles, and radiators). The control of such external heat sources is known in the art. In some embodiments, the at least one vasoconstrictor is applied topically to significant skin areas excluding the hands and/or feet, while the warming glove and sock devices are fitted and activated on the hands and/or feet respectively.

### Example 1

The graphs in FIG. 1 summarize the results of preliminary experiments conducted on rats. Although there are differences between the thermoregulatory system of rats and humans, the tails and feet pads of rats function as thermoregulatory organs in a similar fashion to human skin. The rat tail skin and feet pads are exposed and not covered by fur. By controlling the blood flow to the tail skin and feet pads, the rat controls some of its heat dissipation to the environment. Only about 30% of the rat heat loss is regulated by the tail—much less than the role of the skin in humans.

Male Wistar rats, each about 400 g mass, were divided into three groups: a control group (C) of 8 rats, a test first group (A) of 7 rats, and a third test group (B) of 5 rats. All rats had test composition applied to the tail skin and the feet pads. All experiments were conducted at 23° C. ambient room temperature. All groups went though an anesthesia period of one hour (t=0 is time of start of anesthesia), and a follow-up period of 3 hours after end of anesthesia. Core body temperature (CBT) and tail skin temperature of the rats was measured using a thermocouple-based digital thermometer with 0.1° C. sensitivity.

Group C, the control group, was given a topical cream containing only base cream (similar to Dermabase™ oil-in-water emulsion base, Paddock Laboratories, Inc., Minneapolis, Minn.) with 10 wt. % penetration enhancer (propylene glycol), but no active vasoconstrictor. For test group A, the cream was identical to that used for the control group, except it contained 6 wt. % epinephrine, a vasoconstrictor. The cream for test group B was the same as that for group A, except that the cream contained 20 wt. % penetration enhancer.

The cream on both control group C and test group A was applied at two times: first, concomitantly with application of anesthesia (t=0), and again at 20 min into anesthesia (t=20 m=⅓ h). CBT and tail temperatures were measured at t=0, 20, 40, 60, 120, 180 and 240 minutes.

Plot lines A and C in FIG. 1A show no significant difference in CBT between groups A and C for the first 40 minutes of the experiment, but thereafter show a full one degree difference is maintained between the CBT of the two groups through the remainder of the experiment. On the other hand, as shown in FIG. 1B, already 20 minutes into the experiment it was observed that the temperature of the tail was higher in the control group than in group A, due to decreased blood flow to the skin of the tails of the rats in group A due to vasoconstriction in the tail skin in this group. As application of the anesthetic was discontinued one hour into the experiment, at which point the rats were woken, these results indicate that the vasoconstrictor began to affect the blood vessels in the region to which the vasoconstrictor was applied soon after application, and that the effect of vasoconstrictor application on CBT lasted for at least 3 hours after anesthesia. The results also suggest that in some embodiments, it may be desirable to apply the vasoconstrictor to the skin of the patient 20 to 30 minutes prior to the administration of general anesthetic.

In test group (B), two changes were made to the experimental procedure. First, the concentration of penetration enhancer (propylene glycol) in the cream was doubled to 20 wt. %. Second, the time of first cream application was done at 30 min before anesthesia (t=−30 min) and the second cream application was given with initiation of administration of anesthetic (t=0). The results, shown in the graphs, point to several effects. First, a core temperature difference between control group C and test group B is established already from the start of anesthesia at t=0. Second, there is a stronger effect on CBT in group B than in group A: there is a full 2° C. different in CBT, established within 20 min from anesthesia, and maintained throughout the anesthesia time (t=60 m). Third, there is a rapid fall-off the test group B temperature after 2 h. These results seem to indicate that the increased level of penetration enhancer caused a rapid discharge and absorption of the vasoconstrictor. In contrast, the lower level of penetration enhancer in test group A led to slower release of the vasoconstrictor, giving rise to longer and more stable duration of vasoconstrictor activity in the skin.

### Example 2

### Norepinephrine Bitartrate

This experiment was similar to the experiment of Example 1, although Sprague Dawley albino rats were used. Also, the thermometer used was good for rectal measurements but less fitting for skin measurements than the thermometer used in Example 1; hence, while the core temperature measurements are precise, the tail temperature measurements should be taken as indicative of relative effects but not absolute temperature. The test cream composition was 10% Norepinephrine vasoactive substance, 10% penetration enhancer (propylene glycol), and 80% conventional base cream.

All rats were subjected to one hour pre-anesthesia time (t=−60 to t=0), one hour anesthesia (t=0 to t=60), and 4 hours post anesthesia (t=60 to t=300). The appropriate cream was applied to the tail, feet, and ears skin surface of each rat. There were four rats in each test/control group. Vasoconstrictor cream was applied to the test group rats 3 times, at t=−60, t=−30, and t=0 min before anesthesia. Core body temperature was measured rectally. Rat tail skin temperature was measured at around the mid-length of the tail by press touching the thermometer tip to the skin surface.

The results of the experiment are displayed graphically in FIGS. 2A and 2B (black lines=test group, gray lines=control group). A difference in core body temperature can be observed starting about 20 min into anesthesia. The test group core body temperature is about 1-1.5° C. higher than the control group core body temperature for the remaining anesthesia period and for 60 min post anesthesia.

It is also observed that at about 90 min post anesthesia, the control group temperature goes above the test group temperature, and that at the same time, the tail temperatures are also reversed. The higher/lower skin temperature is evidence for higher/lower degrees of vasodilatation. Thus, although it is not clear at present why this reversal effect is observed with norepinephrine, the results support the claimed method, in that the core temperature is affected by using topical vasoactive substances, where hypothermia during anesthesia is reduced by implementation of a vasoconstrictor action, and conversely hypothermia is increased by vasodilatation effect of topical substances to the skin.

From both the skin and core temperature we conclude that the topical skin Norepinephrine pronounced vasoconstrictor action (leading to cooler tail skin temperature) started about 80 min after the first application of cream, which is slower than the 20-30 min response time of the 6% Epinephrine cream used in Example 1.

Interestingly, according to the literature (Vetrivelan et al., Neural Plasticity 10(4), 267-278 (2003)), injection of methoxamine, an α₁ adrenergic receptor agonist, into the preoptic area of adult male Wistar rat brains resulted in hypothermia.

### Example 3

### Experiment in Pigs

A composition similar to that of Example 1, containing 6 wt. % epinephrine as the vasoconstrictor and 10 wt. % propylene glycol as penentration enhancer was prepared. The same composition, but without the epinephrine, was prepared for use as a control. Five pigs, each weighing 55-65 kg, were divided into two groups, three in the experimental group and two in the control group. The cream was administered 40 minutes before the onset of anesthesia (t=−40 min) and again with the commencement of general anesthesia (t=0). The room temperature was 20° C., with +/−1° C. fluctuations due to the thermostat of the air conditioner. The pigs were not covered with anything other than the cream, with which there were covered over their entire skin surface. Core body temperature was measured at t=−40, 0, 30, 50, 70, 100 and 130 min. The results, expressed as the change in temperature, are shown in FIG. 3. As can be seen, in the control group, represented by diamond, the average decrease in CBT was 2.25° C. after 130 minutes; over this same span, the CBT of the treated pigs decreased on average by only 0.7° C.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the general combination of parts that perform the same functions as exemplified in the embodiments, and includes both combinations and sub-combinations of the various features described hereinabove as well as variations and modifications thereof, which would occur to persons skilled in the art upon reading the foregoing description.

We claim:

1-125. (canceled)

126. A method for treating cancer, comprising inducing hyperthermia in the patient by, at least in part, administering to a cancer patient at least one vasoconstrictor.

127-191. (canceled)

192. A method for treating, preventing or delaying the onset of anesthetic hypothermia, which comprises administering to a patient who is under general anesthetic or is about to be put under general anesthetic an amount of at least one vasoconstrictor effective to treat, prevent or delay the onset of anesthetic hypothermia.

193. (canceled)

194. A method according to claim 192, wherein the at least one vasoconstrictor is provided in a pharmaceutical composition.

195. A method according to claim 194, wherein pharmaceutical composition is a dermally administrable pharmaceutical composition.

196-199. (canceled)

200. A method according to claim 195, wherein the dermally administrable pharmaceutical composition is applied to at least 30% of the patient's skin.

201-205. (canceled)

206. A method according to claim 194, wherein the pharmaceutical composition containing at least 1% by weight of the at least one vasoconstrictor.

207. A method according to claim 206, wherein the pharmaceutical composition contains at least 2% by weight of the at least one vasoconstrictor.

208-212. (canceled)

213. A method according to claim 207, wherein the pharmaceutical composition contains at least 20% by weight of the at least one vasoconstrictor.

214-215. (canceled)

216. A method according to claim 192, wherein at least two vasoconstrictors are used, each of the at least two vasoconstrictors (a) having peak effectiveness at different times after administration, (b) exerting its vasoconstrictive effect via a different mechanism and/or (c) having a different duration of effectiveness after administration.

217. A method according to claim 195, wherein at least two vasoconstrictors are used, each of the at least two vasoconstrictors (a) having peak effectiveness at different times after administration, (b) exerting its vasoconstrictive effect via a different mechanism, and/or (c) having a different duration of effectiveness after administration.

218. (canceled)

219. A method according to claim 192, wherein the at least one vasoconstrictor is selected from the group consisting of (i) the group consisting of vasoactive agonists, vasopressor agents and vasoconstrictor drugs; (ii) an agent that acts on vasopressin receptors or adrenoreceptors; (iii) a calcium channel agonist; (iv) an agonist of the α₁ adrenergic receptor; (v) alfuzosin, doxazosin, epinephrine, methoxamine, naphazoline, norepinephrine, phenylephrine, prazosin, terazosin, tetrahydrozaline, tamsulosin; (vi) an agonist of the 5HT₁B_{A}D receptor; (vii) almotriptan, avitriptan, frovatriptan, oxidesumitriptan, rizatriptan, zolmitriptan; (viii) chlorpheniramine, ethylnorephinephrine, mephenterine, metaraminol, oxymetazoline, oxymetazoline, phenylpropanolamine, potassium chloride, pseudoephidrine, propylhexadrine; (ix) ephedrine, angiotensin and vasopressin; (x) tetrahydrozoline HCl 0.05%, naphazoline HCl 0.03%, oxymetazoline HCl 0.025%; (xi) a vasoconstrictor extract selected from the group including ephedra sinica (ma huang), polygonum bistorta (bistort root), hamamelis virginiana (witch hazel), hydrastis canadensis (goldenseal), lycopus virginicus (bugleweed), aspidosperma quebracho (quebracho bianco), cytisus scoparius (scotch broom), guava extract, ellagic acid, caffeine, peppermint extract, chamomile oil, and cypress; (xii) an agent that positively affects the McKenzie vasoconstrictor assay; (xiii) topical corticosteroids, hydrocortisone, cortisol, synthetic corticosteroids, betametasone, fluticasone, mometasone; (xiv) antagonists of the β₂ adrenergic receptor.

220. A method according to claim 192, wherein the at least one vasoconstrictor or at least one vasoconstrictor in the mixture of vasoconstrictors is selected from the group consisting of methoxamine, methylnorepinephrine, oxymetazoline, phenylephrine, metaraminol, 4-NEMD, clonidine, guanfacine, guanabenz, guanoxabenz, guanethidine, xylazine, methyldopa, apraclonidine, brimonidine, detomidine, dexmedetomidine, lofexidine, romifidine, tizanidine, xylometazoline, amidephrine, amitraz, anisodamine, ergotamine, indanidine, medetomidine, mephentermine, midodrine, mivazerol, naphazoline, norfenefrine, octopamine, phenylpropanolamine, rilmenidine, synephrine, talipexole, tetrahydrozoline, xylometazoline, dobutamine, dopamine, denopamine, xamoterol, salbutamol, levosalbutamol, fenoterol, terbutaline, pirbuterol, procaterol, bitolterol, rimiterol, carbuterol, tulobuterol, reproterol, dopexamine, arformoterol, bambuterol, clenbuterol, formoterol, salmeterol, orciprenaline, metaproterenol, ritodrine, hexoprenaline, indacaterol, amibegron, solabegron, arbutamine, befunolol, isoxsuprine, nylidrin, oxyfedrine, prenalterol, ractopamine, bromoacetylalprenololmenthane, broxaterol, cimaterol, higenamine, mabuterol, methoxyphenamine, tretoquinol, zinterol isoprenaline, isoproterenol, epinephrine, norepinephrine, cirazoline, etilefrine, amphetamine, tyramine, ephedrine, pseudoephedrine, cocaine, allobarbital, amobarbital, aprobarbital, barbital, butobarbital, cyclobarbital, ethallobarbital, heptabarbital, hexobarbital, methohexital, pentobarbital, phenobarbital, proxibarbal, reposal, secobarbital, talbutal, thiopental, vinylbital, vinbarbital, brotizolam, cinolazepam, doxefazepam, estazolam, flunitrazepam, flurazepam, flutoprazepam, loprazolam, lormetazepam, nitrazepam, nimetazepam, midazolam, quazepam, temazepam, triazolam CL-218872, eszopiclone, indiplon, necopidem, pazinaclone, ROD-188, saripidem, suproclone, suriclone, SX-3228, U-89843A, U-90042, zaleplon, zolpidem, zopiclone, glutethimide, methyprylon, pyrithyldione afloqualone, cloroqualone, diproqualone, etaqualone, mebroqualone, mecloqualone, methaqualone, methylmethaqualone acebrochol, allopregnanolone, alphadolone, alphaxolone, ganaxolone, hydroxydione, minaxolone, Org 20599, tetrahydrodeoxycorticosterone, dexmedetomidine, lofexidine, medetomidine, romifidine, tizanidine, xylazine agomelatine, melatonin, ramelteon, doxylamine, hydroxyzine, diphenhydramine, bromodiphenhydramine, carbinoxamine, orphenadrine, niaprazine, phenyltoloxamine, propiomazine, pyrilamine, scopolamine, aceburic acid, gamma-amino-beta-hydroxybutyric acid (GABOB), gamma-hydroxybutyric acid (GHB), sodium oxybate, Xyrem®, gamma-butyrolactone (GBL), 1,4-butanediol, 3-chloropropanoic acid, acetylglycinamide chloral hydrate, chloral hydrate, chloralodol, dichloralphenazone, paraldehyde, petrichloral, centalun, ethchlorvynol, ethinamate, hexapropymate, methylpentynol, meprobamate, carisoprodol, tybamate, methocarbamol, 2-methyl-2-butanol, acecarbromal, apronal, bromisoval, carbromal, clomethiazole, embutramide, etomidate, gaboxadol, loreclezole, mephenoxalone, sulfonmethane, trichloroethanol, triclofos, valerian, valnoctamide and trazadone.

221. A method according to claim 192, wherein the at least one vasoconstrictor is selected from the group consisting of α₁ agonists and β₂ blockers.

222. A method according to claim 192, wherein the at least one vasoconstrictor is selected from the group consisting of epinephrine, norepinephrine, and pharmaceutically acceptable salts thereof.

223. A method according to claim 195, wherein the dermally administrable pharmaceutical composition further comprises at least one of the group consisting of an antiseptic, antibiotic, antimycotic, an antiviral compound, and a penetration enhancer.

224-226. (canceled)

227. A method according to claim 192, wherein the at least one vasoconstrictor is administered in conjunction with at least one of (a) a penetration enhancer, (b) heat from an external source, and (c) a thermogenic substance.

228. A method according to claim 192, wherein one vasoconstrictor is used.

229. A method according to claim 192, wherein more than one vasoconstrictor is used.

230. A method according to claim 229, wherein a mixture of vasoconstrictors is used.

231-274. (canceled)

275. A method according to claim 230 wherein the at least one vasoconstrictor is present as a mixture of vasoconstrictors.
